(19) United States
(12) Patent Application Publication
(10) Pub. No.: US 2019/0150953 A1
(43) Pub. Date: May 23, 2019

(54) Device for Creating Pilot Hole to Access Cancellous Bone

(71) Applicant: Avitus Orthopaedics, Inc., Farmington, CT (US)

(72) Inventors: Maxim Budyansky, Farmington, CT (US); Douglas Pattison, East Hartford, CT (US); Charles R. Satti, III, North Branford, CT (US); Neil Shah, Farmington, CT (US)

(73) Assignee: Avitus Orthopaedics, Inc., Farmington, CT (US)

(21) Appl. No.: 16/253,820

(22) Filed: Jan. 22, 2019

Related U.S. Application Data
(63) Continuation-in-part of application No. PCT/US2017/042611, filed on Jul. 18, 2017.
(60) Provisional application No. 62/363,896, filed on Jul. 19, 2016.

(51) Int. Cl. A61B 17/16; A61B 17/17

(52) U.S. Cl. CPC A61B 17/1615; A61B 17/17; A61B 17/1637

(57) ABSTRACT

Devices, systems and methods are provided for penetrating through cortical layers of bone to expose cancellous bone. Exemplary bone-cutting devices include a cutting tip configured to penetrate bone; and a holder adapted to be associated with the cutting tip. The cutting tip generally includes (i) a first portion that functions to anchor the cutting tip relative to a bone substrate, and (ii) one or more flutes that function to remove bone fragments from the bone substrate. The bone cutting device also generally defines a stop feature to control the depth of insertion of the cutting tip relative to the bone substrate. Clinical advantages are realized, e.g., in instances where a hole is needed to be made in a bone anatomy.

### CROSS-REFERENCE TO RELATED APPLICATION(S)

This application claims priority benefit to International Patent Application No. PCT/US2017/042611, filed on Jul. 18, 2017, which itself claims priority benefit to a U.S. provisional patent application entitled “Device for Creating Pilot Hole to Access Cancellous Bone,” which was filed on Jul. 19, 2016, and assigned Ser. No. 62/363,896. This application is also related to and incorporates by reference a non-provisional patent application entitled “Bone Harvesting,” filed on Oct. 24, 2014, and patented as U.S. Pat. No. 9,833,248. The entire contents of the foregoing patent applications are herein incorporated by reference.

### TECHNICAL FIELD

The present invention is directed to devices/systems/methods for creating an opening for accessing bone and other internal structures/materials. For example, the disclosed device/system and method can be utilized for penetrating through cortical layers of bone to expose cancellous bone.

### BACKGROUND ART

Current drill bits used as bone hole creators have several drawbacks that include, inter alia, a lack of tactile feedback from the drill bit, an inability to anchor the drill bit and prevent the bit from slipping off curvy bone surfaces, and a lack of depth control during drilling. The device, systems and methods of the present application address the noted shortcomings and limitations.

### SUMMARY

The present invention discloses devices/systems/methods that can be utilized for penetrating through cortical layers of bone to expose cancellous bone. The disclosed invention offers several clinical advantages for instances where a hole is needed to be made in a bone anatomy. Some clinical scenarios where holes are required include, but are not limited to; whenever a surgeon needs to harvest cancellous bone or marrow, as a starter hole for an implant or for screw placement.

The disclosed device features a triangular center point that offers significant tactile feedback, especially in minimally invasive instances of trying to gain access to a boney anatomy. This allows the user to blindly (i.e., without visibly seeing the tip or bone) feel how much bone there is near the cutting end to gauge centering of the instrument. The triangular center point can stab the bone to anchor the instrument prior to creating the hole, thereby circumventing a common drawback of drill bits that have a tendency to slip on curvy bone surfaces. This feature increases the safety of the procedure.

The disclosed device/system may further feature a depth control feature called the stop feature that prevents over-insertion of the tool. A common drawback to many circular hole cutting devices is a lack of depth control which can lead to perforations in boney anatomies (i.e., puncturing through an unintended area of cortical bone). This invention prevents this undesired drawback with the addition of a stop feature. The design of the flattened cutting blade offers controlled cutting that gradually shaves off layers of bone to create the hole in a bone region.

The disclosed device/system may further feature a cortical cap removal device that can utilize a continuous tapered cutting feature or several cutting flutes in either a rotational motion, translational motion, or a combination thereof, to create and preserve a cortical cap that can be reinserted into the host bone, if desired and/or necessary. A potential drawback to hole drilling is a requirement to fill the hole; with the disclosed cortical cap removal device, the cortical cap is preserved and can be reinserted, thereby overcoming this potential drawback. Further, as is discussed above, the disclosed cortical cap removal device may advantageously feature a stop feature so as to overcome an undesired drawback of lack of depth control.

Additional features, functions and benefits of the disclosed device/system and method will be apparent from the description which follows, particularly when read in conjunction with the accompanying figures.

### BRIEF DESCRIPTION OF THE DRAWINGS

A more complete understanding of the present disclosure and advantages thereof may be acquired by referring to the following description in consideration with the accompanying drawings, in which like reference numbers indicate like features.

FIG. 1A is a front view of a device/system for creating a pilot hole (“pilot hole creator”) according to the present disclosure.

FIG. 1B is a front detailed view of a cutting tip attached (either fixedly or detachably) to or integrally formed with a pilot hole creator according to the present disclosure. For discussion purposes, the various approaches to combining a cutting tip and a pilot hole creator are referenced by the term “associated with” in the disclosure which follows, such verbiage expressly encompassing the various ways in which the disclosed cutting tip may be attached to or formed with the disclosed pilot hole creator.

FIG. 2 is a bottom view of FIG. 1A showing a cutting tip associated with a pilot hole creator according to the present disclosure.

FIG. 3 is a perspective view of a pilot hole creator according to the present disclosure.

FIG. 4A is a perspective view of a cutting tip associated with a pilot hole creator according to the present disclosure.

FIG. 4B is a perspective view of a cutting tip associated with a pilot hole creator according to the present disclosure.

FIG. 4C is a front view of a cutting tip associated with a pilot hole creator according to the present disclosure.

FIG. 5 is a front view of a pilot hole creator with a manual T-handle according to the present disclosure.

FIG. 6A is a front view of a pilot hole creator according to the present disclosure.

FIG. 6B is a front detailed view of a cutting tip associated with a pilot hole creator according to the present disclosure.

FIG. 7A is a front view of a pilot hole creator with an un-rotated T-handle according to the present disclosure.

FIG. 7B is a front view of a pilot hole creator with a rotating T-handle according to the present disclosure.

FIG. 7C is a front view of a pilot hole creator with a rotated T-handle according to the present disclosure.

FIG. 8A is a front view of a pilot hole creator with a slidable handle covering the cutting tip, according to the present disclosure.

FIG. 8B is a front view of a pilot hole creator with a sliding handle, according to the present disclosure.

FIG. 8C is a front view of a pilot hole creator with a locked handle, according to the present disclosure.

FIG. 9A is a front view of a packaged pilot hole creator according to the present disclosure.

FIG. 9B is a front view of an unpackaged pilot hole creator with a slidable handle covering the cutting tip, according to the present disclosure.

FIG. 9C is a front view of an unpackaged pilot hole creator with a sliding handle, according to the present disclosure.

FIG. 9D is a front view of an unpackaged pilot hole creator with a locked handle, according to the present disclosure.

FIG. 10 is a perspective view of a removable cutting tip according to the present disclosure.

FIG. 11 is a front view of a T-handle holder with a removable cutting tip, according to the present disclosure.

FIG. 12A is a front view of a cutting tip preparing to interface with bone layering, according to the present disclosure.

FIG. 12B is a front view of a cutting tip interfacing with bone layering, according to the present disclosure.

FIG. 13A is a front view of a countersink cutting tip preparing to interface with bone layering, according to the present disclosure.

FIG. 13B is a front view of a countersink cutting tip interfacing with bone layering, according to the present disclosure.

FIG. 13C is a front view of a countersunk hole in bone layering, according to the present disclosure.

FIG. 14A is a front view of a countersunk hole in bone layering with a bone harvesting device, according to the present disclosure.

FIG. 14B is a front view of a straight hole in bone layering with a bone harvesting device, according to the present disclosure.

FIGS. 15A and 15B are side views of an alternative pilot hole creator embodiment with an alternative stop feature for controlling penetration depth, according to the present disclosure.

FIG. 16A is a perspective view of an alternate pilot hole creator embodiment for removing and preserving a cortical cap, according to the present disclosure.

FIG. 16B is a front view of an alternate pilot hole creator embodiment for removing and preserving the cortical cap, according to the present disclosure.

FIGS. 17A and 17B depict insertion of a tapered cortical cap remover and the preserved cortical cap associated therewith, according to the present disclosure.

FIG. 18A is a perspective view of an alternate pilot hole creator embodiment for removing and preserving a cortical cap, according to the present disclosure.

FIG. 18B is a front view of an alternate pilot hole creator embodiment for removing and preserving a cortical cap, according to the present disclosure.

FIGS. 19A and 19B depict insertion of a cortical cap remover and the preserved cortical cap associated therewith, according to the present disclosure.

FIG. 20A is a perspective view of an alternate pilot hole creator embodiment for removing and preserving a cortical cap, according to the present disclosure.

FIG. 20B is a front view of an alternate pilot hole creator embodiment for removing and preserving a cortical cap, according to the present disclosure.

FIGS. 21A and 21B depict insertion of a cortical cap remover and the preserved cortical cap associated therewith, according to the present disclosure.

FIGS. 22A to 22C depict an alternative pilot hole creator systems according to the present disclosure.

FIGS. 23A to 23D depict a further alternative pilot hole creator systems according to the present disclosure.

FIGS. 24A to 24D depict a guide system in an exemplary use case according to the present disclosure.

FIGS. 25A and 25B depict ancillary features associated with exemplary guide systems according to the present disclosure.

FIGS. 26-28 depict exemplary systems for fastening a cortical cap relative to a cortical hole according to the present disclosure.

FIGS. 29A and 29C depict exemplary angled cutting devices according to the present disclosure.

FIG. 29B depicts an exemplary anchor spike according to the present disclosure.

FIGS. 30A to 30F depict an exemplary use case for an angled carver according to the present disclosure.

FIGS. 31A, 31C and 31D depict an alternative angled carver system according to the present disclosure.

FIG. 31B depicts an exemplary anchor spike according to the present disclosure.

FIGS. 32A to 32D depict an alternative angled carver system according to the present disclosure.

FIGS. 33A to 33E depict an alternative angled carver system with a retractable carving blade according to the present disclosure.

FIGS. 34A to 34C depict an alternative angled carver system with a retractable carving blade and a ring blade according to the present disclosure.

FIGS. 35A to 35D depict an exemplary system that includes a chisel punch blade with a downward cutter according to the present disclosure.

FIGS. 36A to 36E depict exemplary operation of the chisel punch blade depicted in FIGS. 35A-35D according to the present disclosure.

FIGS. 37A to 37D depict an alternative cutting system with multi-slotted blade according to the present disclosure.

FIGS. 38A to 38E depict exemplary operation of the multi-slotted blade of FIGS. 37A-37D according to the present disclosure.

FIGS. 39A to 39D depict the alternative cutting system with multi-slotted blade of FIGS. 37A to 37D with a central cannulation according to the present disclosure.

FIGS. 40A-40F depict additional concepts for fastening according to the present disclosure.

FIG. 41 depicts an adjustable hole saw according to the present disclosure.

### DETAILED DESCRIPTION OF THE DRAWINGS

As referenced above, the present application relates generally to a pilot hole creator, designed for penetrating through cortical layers of bone to expose cancellous bone, and includes, inter alia, a cutting tip associated with a handle structure. Thus, the cutting tip may be fixedly or detachably mounted with respect to a handle structure, or integrally formed with a handle structure.

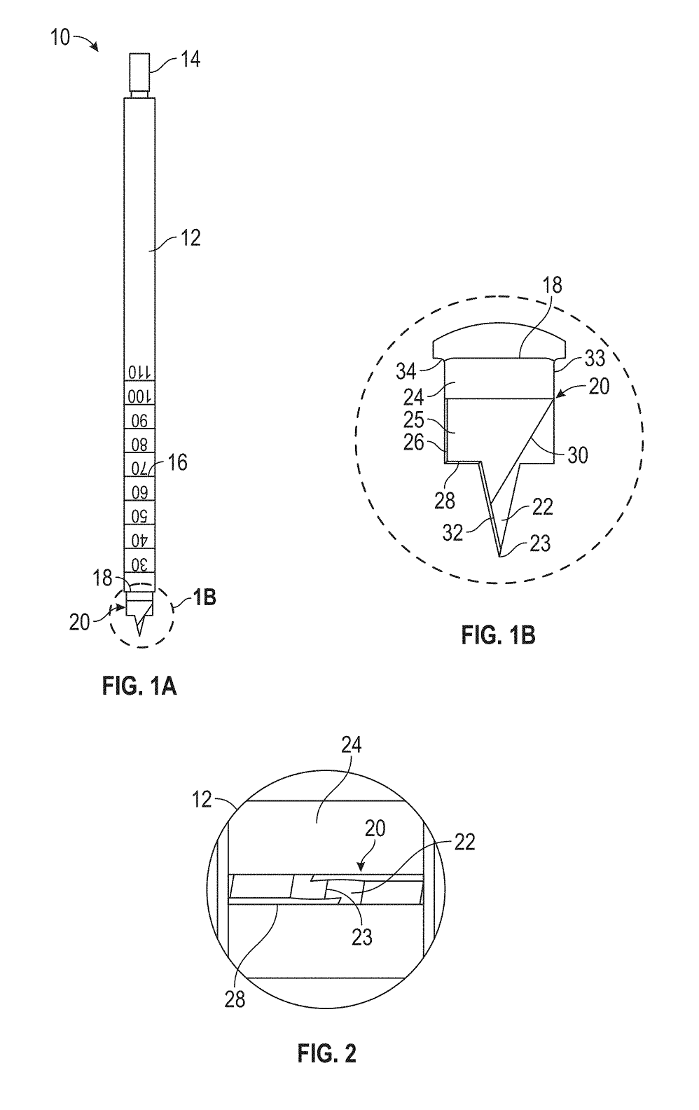

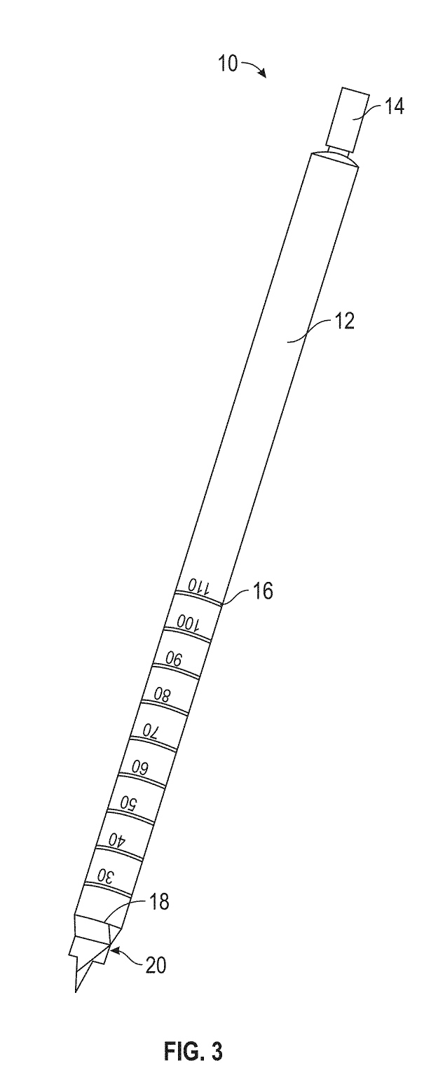

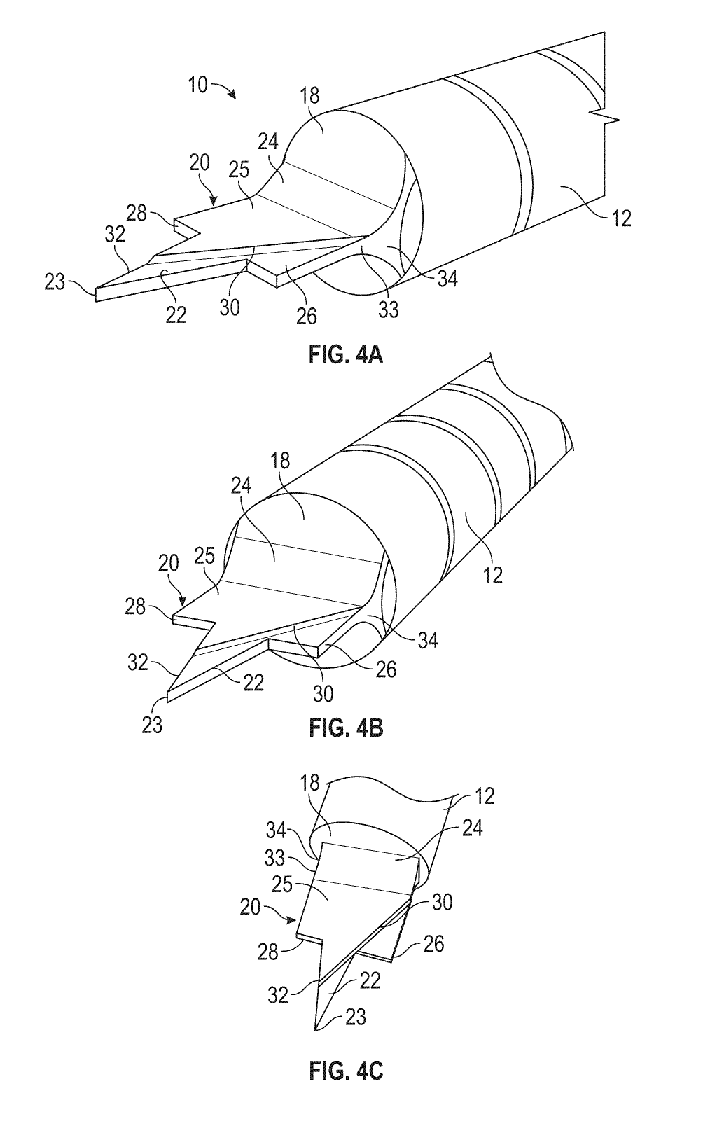

FIGS. 1A-4C illustrate an exemplary pilot hole creator 10 wherein cutting tip 20 is integrated with shaft 12 at surface 18. The manner of integration of the cutting tip/shaft subassembly may take various forms, e.g., a fixed connection, a detachable connection or an integrally-formed subassembly. In the exemplary embodiment of FIGS. 1A-4C, the shaft 12 is cylindrical in shape. However, shaft 12 is not limited to a cylindrical geometry; for example, shaft 12 may be formed with an oval/elliptical cross-sectional geometry, a square/rectangular cross-sectional geometry, a polygonal cross-sectional geometry (e.g., pentagonal, hexagonal, etc.) and combinations thereof. The cross-sectional geometry of shaft 12 may be uniform over its length, or be non-constant, e.g., transitioning from one cross-sectional geometry to a second, different cross-sectional geometry (indeed, multiple variations in cross-sectional geometries may be defined along the overall length of shaft 12 according to the present disclosure). The overall length of shaft 12 may vary depending on clinical needs/factors. In exemplary embodiments of the present disclosure, shaft 12 is generally between 0.5″ and 18″ in length, although lengths outside of this exemplary range may be employed as necessary to facilitate specific clinical applications.

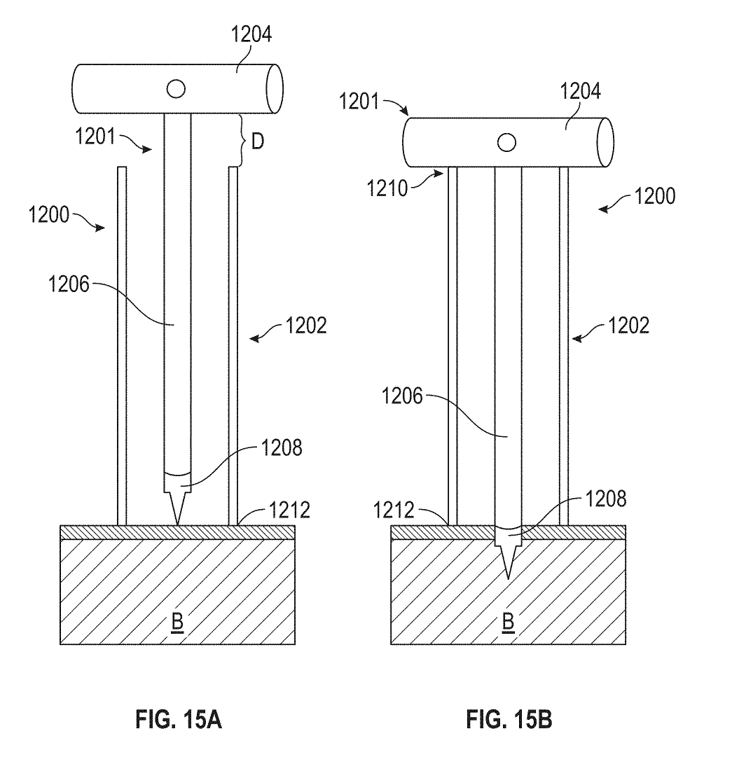

Shaft 12 can be fabricated from any material suitable for insertion into the human body (e.g., metal, plastic and combinations thereof) as will be apparent to someone skilled in the art. Surface 18 is substantially flat and provides a stop feature for cutting tip 20, wherein the cross-section of surface 18 is greater than the width of cutting tip 20, thus limiting the depth of cut to the distance from surface 18 to triangular center point 23 of cutting tip 20, thereby preventing over insertion of the tool. The disclosed stop feature may take various forms and may be positioned at various axial locations along the disclosed device. For example, a stop feature/structure may be positioned between the disclosed cannulated port and the pilot hole creator, although the present disclosure is not limited by or to a specific location and/or structural design for the disclosed stop feature/structure. Indeed, an alternative exemplary design featuring an alternative stop mechanism to limit penetration depth is depicted in FIGS. 15A and 15B, as discussed hereinbelow. Furthermore, as an additional safety feature, shaft 12 may provide gradation marks 16 to assist the user in depicting the depth of cutting tip 20 by referencing the marks 16 against a surface with known distance to the cutting area.

A drill drive system (i.e., a powered drive system) and/or a manual handle is generally affixed (either fixedly or detachably) to feature 14, thereby enabling the user to operate pilot hole creator 10. Of note, the disclosed manual handle may take various forms and geometries. In exemplary embodiments, the manual handle takes the form of a T-handle, although alternative forms/geometries are specifically contemplated according to the present disclosure, e.g., a ball handle, a pistol handle, a bayonet handle, screw driver handle, palm swell design, and the like. Thus, the form/geometry of the disclosed manual handle may take various forms/geometries without departing from the spirit or scope of the present disclosure.

With specific reference to FIGS. 1B, 2 and 4A-4C, two larger fillets 24 extend from surface 25 of cutting tip 20 and interface with surface 18, thereby strengthening the cutting tip 20. An additional two smaller fillets 34 extend from side 33 of surface 24, thereby further strengthening the cutting tip. In one embodiment, cutting tip 20 includes two flutes 30, designed to separate bone/material fragments from a bone substrate to facilitate efficient cutting from the six cutting surfaces 26, 28 and 32. However, cutting tip 20 can include more or less flutes and cutting surfaces, as will be apparent to one skilled in the art.

In exemplary embodiments of the present disclosure, cutting tip 20 can be removed from shaft 12, e.g., when cutting tip 20 is damaged/dull, without requiring the user to purchase a new pilot hole creator 10 every time the cutting tip 20 is no longer serviceable. Thus, a detachable cutting tip 20 offers enhanced economics according to exemplary embodiments of the present disclosure.

As the disclosed device interacts with a bone substrate, the triangular center point 23 of cutting tip 20 offers significant tactile feedback enabling the user to “blindly” (i.e. without visibly seeing the bone) survey the bone and determine the approximate center to create the hole. Furthermore, the triangular center point 23 of cutting tip 20 can be plunged into the bone to anchor the pilot hole creator 10, prior to cutting, so as to avoid slipping off curved bone surfaces.

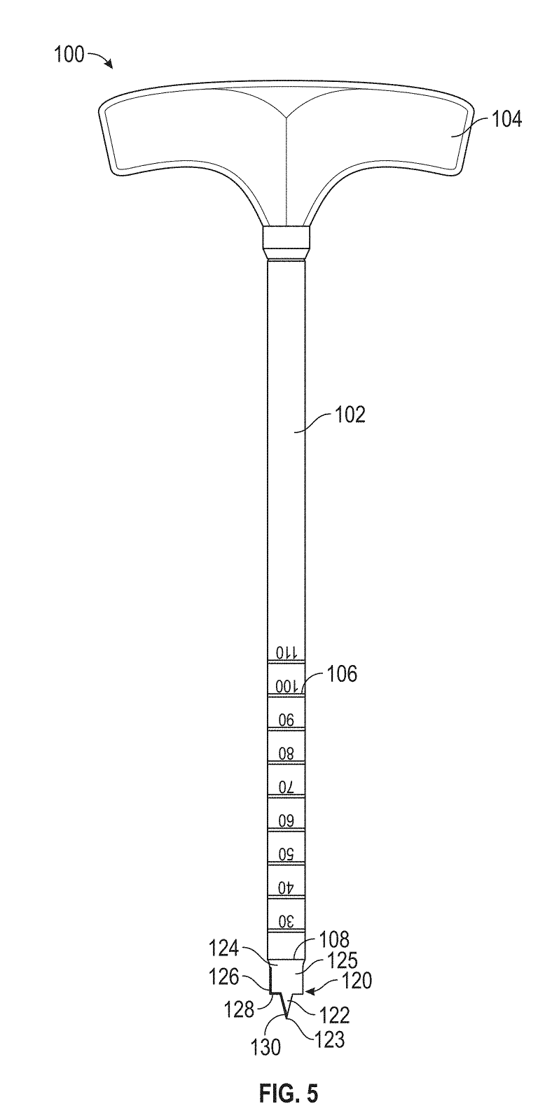

FIG. 5 illustrates another exemplary embodiment according to the present disclosure. Pilot hole creator 100 includes a shaft 102 associated with a manual handle 104. As noted above, the shaft 102 may be fixedly or detachably connected to handle 104, or may be integrally formed therewith. The manual handle 104 may take various forms/geometries, e.g., the T-handle depicted in FIG. 5 or alternative forms/geometries, e.g., e.g., a ball handle, a pistol handle, a bayonet handle, screw driver handle, palm swell design, and the like. Thus, as described above with reference to the embodiment of FIGS. 1-4C, the form/geometry of the disclosed manual handle may take various forms/geometries without departing from the spirit or scope of the present disclosure.

Shaft 102 is cylindrical in shape. However, as with shaft 12 in FIGS. 1-4C, shaft 102 is not limited to a cylindrical geometry; for example, shaft 102 may be formed with an oval/elliptical cross-sectional geometry, a square/rectangular cross-sectional geometry, a polygonal cross-sectional geometry (e.g., pentagonal, hexagonal, etc.) and combinations thereof. The cross-sectional geometry of shaft 102 may be uniform over its length, or be non-constant, e.g., transitioning from one cross-sectional geometry to a second, different cross-sectional geometry (indeed, multiple variations in cross-sectional geometries may be defined along the overall length of shaft 102 according to the present disclosure).

The overall length of shaft 102 may vary depending on clinical needs/factors. In exemplary embodiments of the present disclosure, shaft 102 is generally between 0.5″ and 18″ in length, although lengths outside of this exemplary range may be employed as necessary to facilitate specific clinical applications.

Shaft 102 can be fabricated from any material suitable for insertion into the human body (e.g., metal, plastic or combinations thereof) as will be apparent to someone skilled in the art. Shaft 102 is outfitted with gradation marks 106 functioning to depict the depth of cutting tip 120. Similar to the embodiments discussed above, cutting tip 120 is mounted to surface 108 wherein two larger fillets 124 extend from surface 125 and two smaller fillets (not shown) extend from the side of surface 125. The geometry of cutting tip 120 is substantially similar to cutting tip 20, described above, wherein cutting tip 120 features two flutes (not shown) and six cutting surfaces 126, 128 and 130. Cutting tip 120 can include more or less flutes and cutting surfaces, as will be apparent to one skilled in the art.

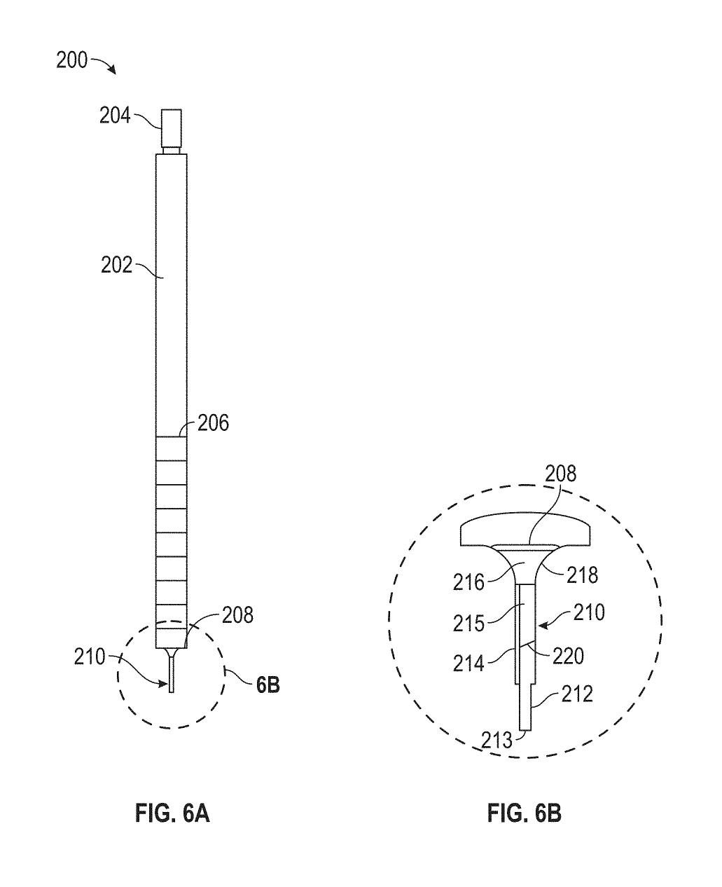

In another exemplary embodiment as depicted in FIGS. 6A and 6B, pilot hole creator 200 includes shaft 202 which is associated with a cutting tip 210 via surface 208. The manner of association between shaft 202/cutting tip 210 may take various forms, as discussed above with reference to previous embodiments. Similarly, shaft 202 may be cylindrical in shape, although alternative geometries are specifically contemplated, e.g., an oval/elliptical cross-sectional geometry, a square/rectangular cross-sectional geometry, a polygonal cross-sectional geometry (e.g., pentagonal, hexagonal, etc.), a substantially flat/planar geometry and combinations thereof. The cross-sectional geometry of shaft 202 may be uniform over its length, or be non-constant, e.g., transitioning from one cross-sectional geometry to a second, different cross-sectional geometry (indeed, multiple variations in cross-sectional geometries may be defined along the overall length of shaft 202 according to the present disclosure).

The overall length of shaft 202 may vary depending on clinical needs/factors. In exemplary embodiments of the present disclosure, shaft 202 is generally between 0.5″ and 18″ in length, although lengths outside of this exemplary range may be employed as necessary to facilitate specific clinical applications.

Shaft 202 can be fabricated from any material suitable for insertion into the human body (e.g., metal, plastic or combinations thereof) as will be apparent to someone skilled in the art.

Surface 208 is substantially flat and provides a stop feature for the cutting tip 210, wherein the cross-section of surface 208 is greater than the width of cutting tip 210 and the depth of cut is limited to the distance from surface 208 to edge 213 of cutting tip 210, thereby preventing over insertion of the tool. Furthermore, as an additional safety feature, shaft 202 provides gradation marks 206 to assist the user in determining the depth of cutting tip 210 by referencing the marks 206 against a surface with known distance to the cutting area.

A drill drive system (i.e., a powered drive system) and/or a manual handle is generally affixed (either fixedly or detachably) to feature 204, thereby enabling the user to operate pilot hole creator 200. Of note, the disclosed manual handle may take various forms and geometries. In exemplary embodiments, the manual handle takes the form of a T-handle, although alternative forms/geometries are specifically contemplated according to the present disclosure, e.g., a ball handle, a pistol handle, a bayonet handle, screw driver handle, palm swell design, and the like. Thus, the form/geometry of the disclosed manual handle may take various forms/geometries without departing from the spirit or scope of the present disclosure.

With specific reference to FIG. 6B, two fillets 216 extend from surface 215 of cutting tip 210 and interface with surface 208, thereby strengthening the cutting tip 210. An additional two fillets 218 extend from side 214 of surface 215, thereby further strengthening the cutting tip. In one embodiment, cutting tip 210 includes two flutes 220, designed to free up bone/material fragments to facilitate efficient cutting from the four cutting surfaces 212 and 214. However, cutting tip 210 can include more or less flutes and cutting surfaces, as will be apparent to one skilled in the art. In another embodiment, cutting tip 210 can be removed from shaft 202 wherein cutting tip 210 can be replaced when damaged or dull without requiring the user to purchase a new pilot hole creator 200 every time the cutting tip 210 is no longer serviceable. The flattened feature 213 of cutting tip 210 offers controlled cutting that gradually shaves off layers of bone to create a hole, providing more control over the depth.

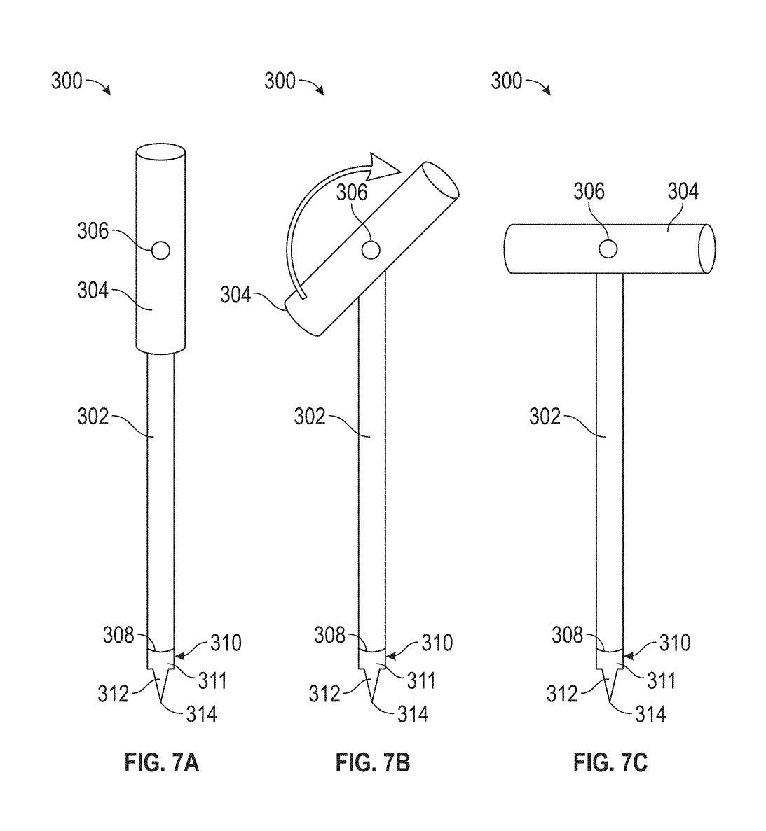

With reference to FIGS. 7A-7C, exemplary pilot hole creator 300 includes cutting tip 310, which is substantially similar to cutting tip 20, described above, wherein cutting tip 310 is associated with surface 308 of shaft 302. Although not pictured, the cross-section of shaft 302 can be larger than the width of cutting tip 310, thereby providing an advantageous stop feature, wherein the maximum hole depth is the distance from triangular center point 314 of cutting tip 310 to surface 308 of shaft 302.

With specific reference to FIG. 7A, manual handle 304 (shown as a T-handle, although alternative handle designs/geometries may be employed according to the present disclosure) is in the unrotated/storing position. As illustrated in FIG. 7B, T-handle 304 rotates around the axis of pin 306 from a substantially vertical position to a final position perpendicular to shaft 302, as illustrated in FIG. 7C.

In one exemplary embodiment, T-handle 304 rotates in a clockwise motion. In another exemplary embodiment, T-handle 304 rotates in a counterclockwise motion. In either embodiment, T-handle 304 may advantageously lock into place once it is perpendicular to shaft 302, as illustrated in FIG. 7C. The locking mechanism (not shown) can include one or more detents, a tightening mechanism (e.g., based on tightening of pin 306) or another locking mechanism that would be readily apparent to one skilled in the art based on the disclosure herein. The locking mechanism may be permanent, or it may be reversible, allowing the T-handle 304 to rotate back to its original substantially vertical position as illustrated in FIG. 7A.

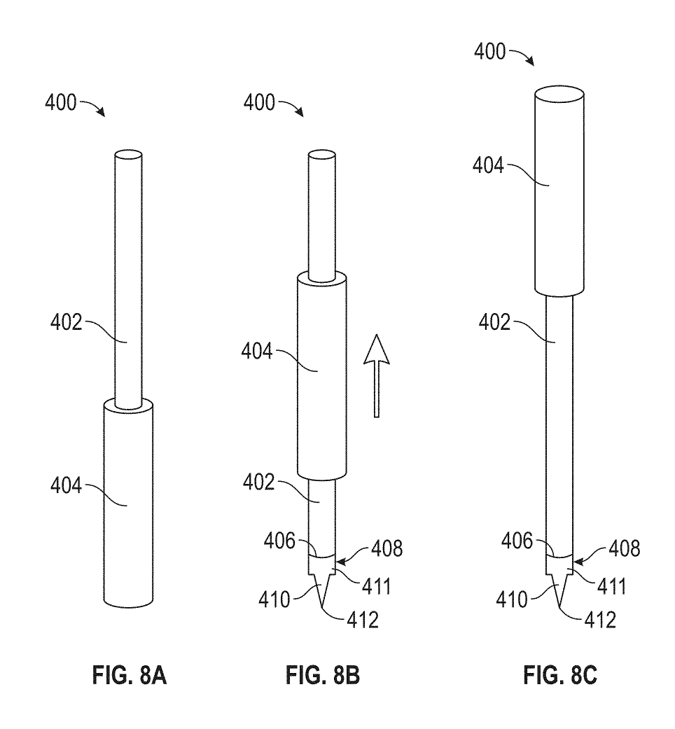

With reference to FIGS. 8A-8C, exemplary pilot hole creator 400 includes cutting tip 408, which is substantially similar to cutting tip 20, described above, wherein cutting tip 408 is associated with surface 406 of shaft 402. The cross-section of shaft 402 is substantially equal to the width of cutting tip 408, thereby facilitating creation of a hole that extends deeper than the distance from surface 406 of shaft 402 to the triangular center point 412 of cutting tip 408. However, although not pictured, the cross-section of shaft 402 can be larger than the width of cutting tip 408, thereby defining a stop feature, wherein the maximum hole depth is the distance from triangular center point 412 of cutting tip 408 to surface 406 of shaft 402.

With specific reference to FIG. 8A, handle 404 is substantially covering the cutting tip (not shown), thereby protecting the cutting tip (not shown) from being damaged during transit/storage and protecting the user from being injured. As is evident from FIGS. 8B and 8C, handle 404 slides axially relative to shaft 402 from cutting tip 408 to the opposite end (i.e., top) of shaft 402, wherein upon reaching the top of shaft 402, handle 404 “clicks” into place, thereby creating a “screw driver type” handle for the user. The locking mechanism (not shown) can include one or more detents, a pin/bolt mechanism or another locking mechanism that would be readily apparent to one skilled in the art based on the disclosure herein. The locking mechanism may be permanent, or it may be reversible, allowing handle 404 to slide axially back to its original position as illustrated in FIG. 8A.

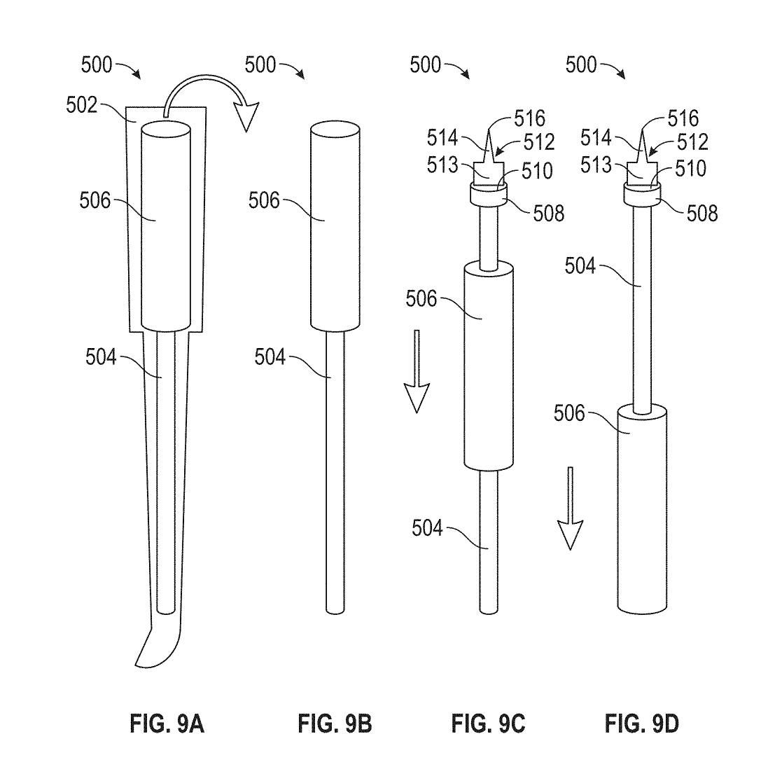

With reference to FIG. 9A, exemplary pilot hole creator 500 is encased in packaging 502 wherein handle 506 is substantially covering the cutting tip (not shown), as described above. Packaging 502 can be any flexible or non-flexible enclosure that is acceptable for medical devices (e.g., accommodative of conventional sterilization procedures), as will be apparent to persons skilled in the art. FIG. 9B illustrates the pilot hole creator 500 removed from packaging 502, wherein pilot hole creator 500 is substantially similar to pilot hole creator 400, described above. In one embodiment, pilot hole creator 500 can be identical to pilot hole creator 400. In another embodiment, pilot hole creator 500 can include a shaft 504 with a cross-section smaller than cutting tip 512, wherein cutting tip 512 is attached to (or otherwise associated with) surface 510 of collar 508. Surface 510 of collar 508 has a cross-section that is larger than the width of cutting tip 512, thereby providing a stop feature that prevents over insertion of the cutting tip 512, wherein the depth of the hole is limited to the distance from triangular center point 516 of cutting tip 512 to surface 510 of collar 508.

With reference to FIGS. 9C and 9D, handle 506 slides axially relative to shaft 504 from cutting tip 512 to the opposite end (i.e., top) of shaft 504, as described in FIGS. 8B and 8C. Upon reaching the top of shaft 504, handle 506 “clicks” into place, thereby creating a “screw driver type” handle for the user. The locking mechanism (not shown) can include one or more detents, a pin/bolt mechanism or another locking mechanism that would be readily apparent to one skilled in the art based on the present disclosure. The locking mechanism may be permanent, or it may be reversible, allowing handle 506 to slide axially back to its original position as illustrated in FIG. 9B.

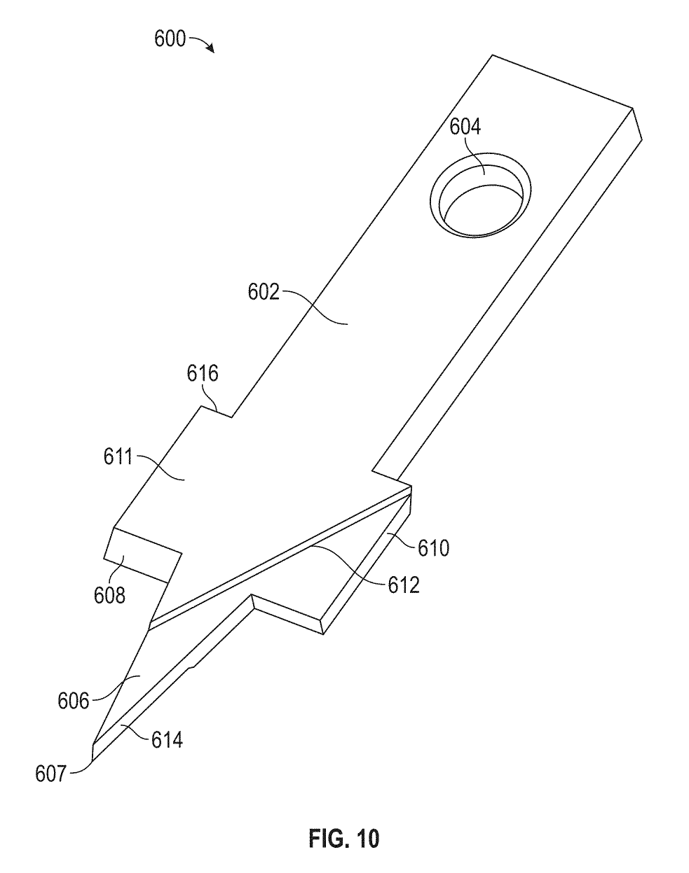

With reference to FIG. 10, exemplary cutting tip 600 includes an attachment interface 604, cutting surfaces 608, 610 and 614, and flute 612. Cutting tip 600 is a disposable accessory that can easily be removed and replaced from a holder. Similar to cutting tip 20, cutting tip 600 includes two flutes 612, designed to remove bone/material fragments from a bone substrate to facilitate efficient cutting from the six cutting surfaces 608, 610 and 614. However, cutting tip 600 can include more or less flutes and cutting surfaces, as will be apparent to one skilled in the art.

Triangular center point 607 of cutting tip 600 offers significant tactile feedback enabling the user to “blindly” (i.e. without visibly seeing the bone) survey the bone and determine the approximate center or location of interest for entry to create the hole. Furthermore, the triangular center point 607 of cutting tip 600 can be plunged into the bone to anchor the pilot hole creator so as to avoid slipping off curved bone surfaces. Cutting tip 600 is not limited to the disclosed embodiment; other embodiments, including cutting tip 210, can be utilized, as will be apparent to persons skilled in the art.

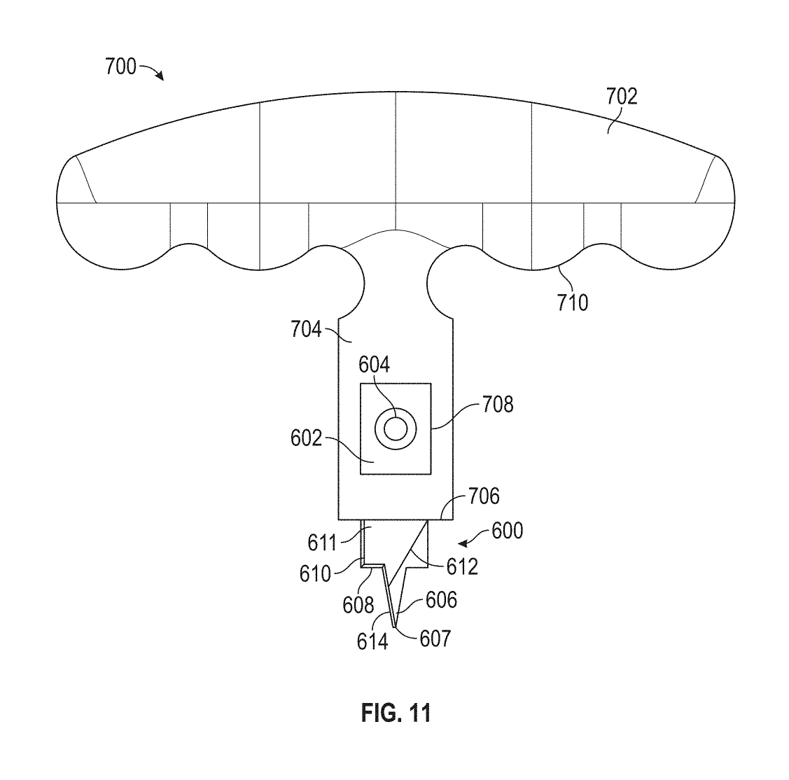

With further reference to FIG. 10 and initial reference to FIG. 11, shaft 602 extends from body 611 wherein the width of shaft 602 is less than the width of body 611, thereby creating surface 616 which abuts device 700 at surface 706. Surface 616 provides an additional point of contact when interfacing with holder 700. In one embodiment, device 700 includes surface 702 for interfacing with a user's palm and sinusoidal-like undulations 710 for a user's fingers. In another embodiment, device 700 can resemble the rotating T-handle of pilot hole creator 300 or the sliding screwdriver-like handle of pilot hole creator 400 and 500. Extending perpendicular from finger grip 710 is holder 704, which includes attachment interface mechanism 708 and cutting tip slot (not shown).

In operation/use, shaft 602 of cutting tip 600 is inserted into cutting tip slot (not shown) located on surface 706 of device 700. Cutting tip slot (not shown) can be substantially larger or minimally larger than shaft 602. Cutting tip 600 is inserted a depth equal to the distance from surface 616 to the opposing edge of shaft 602. Attachment interface 604 of cutting tip 600 engages with attachment interface mechanism 708 of device 700, thereby creating a secure connection. Attachment interface 604 is depicted as being substantially circular, however, various interface designs can be utilized (e.g., slot, square, triangle, or notches on the side of shaft 602), as will be apparent to persons skilled in the art. Attachment interface mechanism 708 will have features that will capture the design of attachment interface 604, such as detents, a pin, a magnet, among others, as will be apparent to persons skilled in the art. When in use, surface 706 acts as a stop feature thereby limiting the hole depth to the distance between triangular center point 607 of cutting tip 600 and surface 706 of device 700. The operation of cutting tip 600 is not limited to the disclosed design of device 700, other devices can be utilized, as will be apparent to persons skilled in the art.

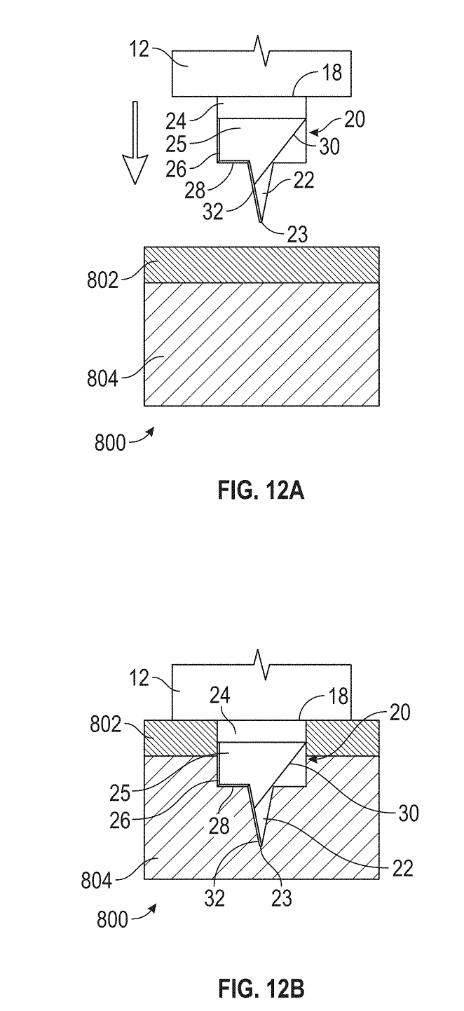

With reference to FIG. 12A, cutting tip 20 is hovering over the bone 800 wherein triangular point 23 of cutting tip 20 would first contact/penetrate the cortical bone region 802 before entering the cancellous bone region 804. Cutting tip 20 may be manually rotated by the user or rotation may be powered by a drive mechanism. The rate of rotation (i.e., rpm) is generally controlled so as to provide substantially controlled introduction to the bone substrate. Note, although only cutting surfaces on one side of cutting tip 20 are referenced, cutting tip 20 can have at least one additional set of cutting surfaces. The total number of cutting surfaces is generally selected based on clinical needs/objectives, as will be readily apparent to persons skilled in the art based on the present disclosure.

As is evident from FIG. 12B, the depth of the hole created in the bone 800 is limited to the distance from surface 18 of shaft 12 to triangular center point 23 of cutting tip 20, wherein surface 18 advantageously acts as a stop feature. In this embodiment, a straight hole was created; however, other hole designs and entry paths are possible according to the present disclosure, as will be apparent to those skilled in the art.

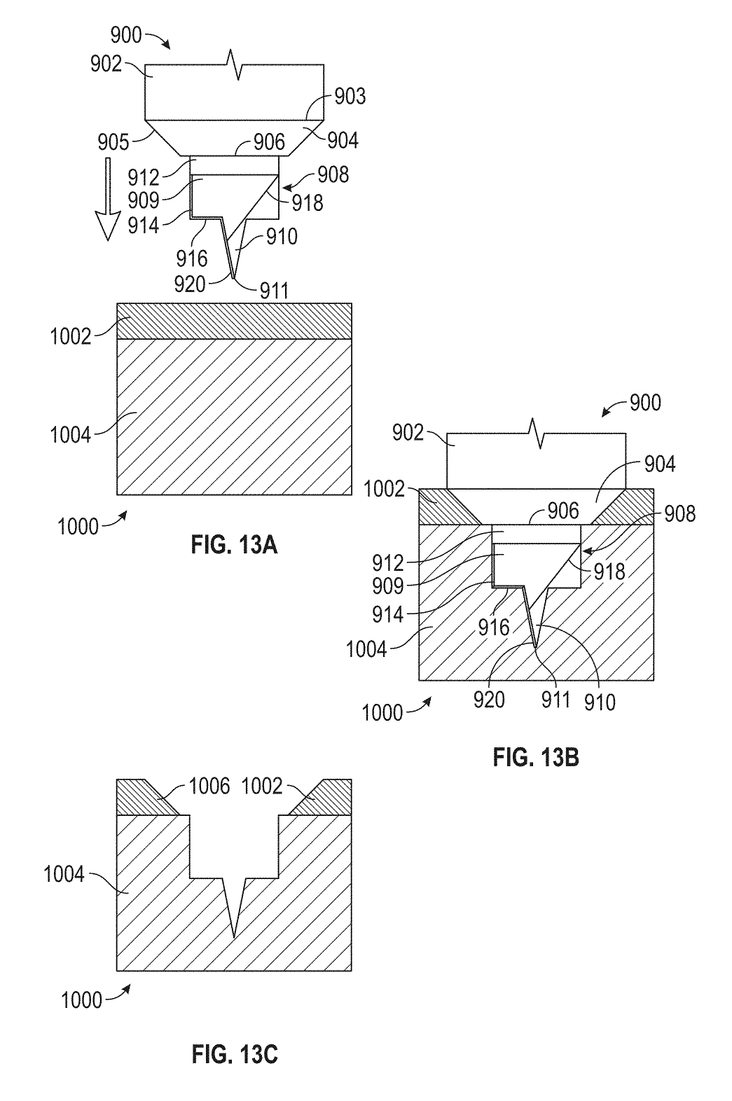

With reference to FIG. 13A, exemplary cutting tip 908 is shown hovering over bone 1000, wherein triangular point 911 of cutting tip 908 would first contact/penetrate the cortical bone region 1002 before entering the cancellous bone region 1004. In addition to the previously described cutting surfaces of cutting tip 908, pilot hole creator 900 also includes a countersink cutting feature 904, with cutting surface 905, for creating a chamfered edge in the bone 1000. Flutes 918 would separate bone/material fragments to ensure an efficient cutting process. Note, although only cutting surfaces on one side of cutting tip 908 are referenced, cutting tip 908 can have at least one additional set of cutting surfaces as described above. The total number of cutting surfaces to be employed are generally selected based on clinical needs/objective, as will be readily apparent to persons skilled in the art based on the present disclosure.

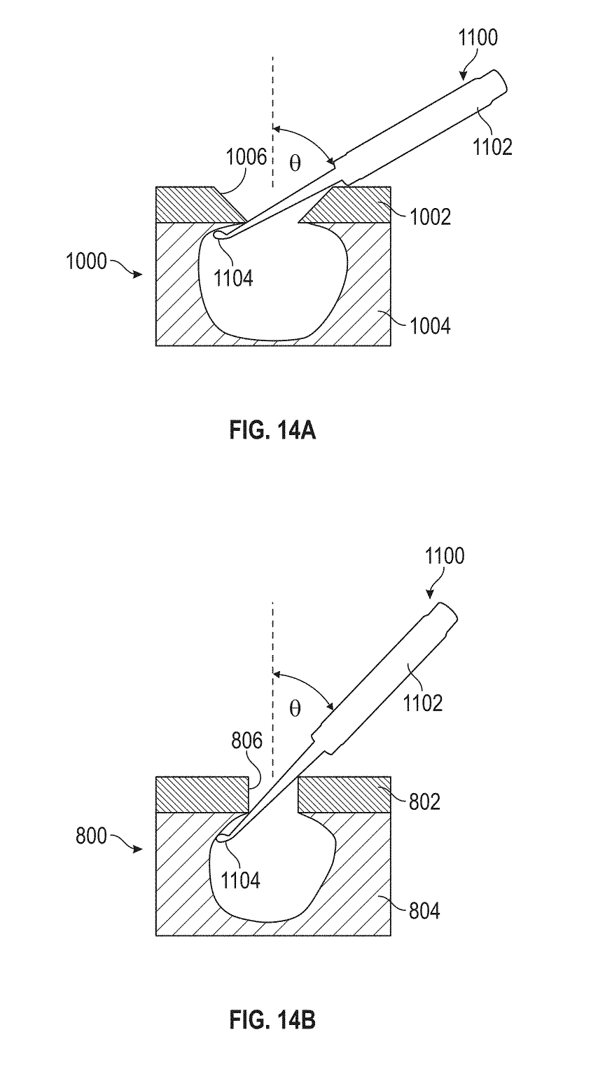

As is evident from FIGS. 13B and 13C, the depth of the hole created in the bone 1000 is limited to the distance from surface 903 of shaft 902 to triangular center point 911 of cutting tip 908. In addition to the straight hole created by cutting tip 908, countersink cutting feature 904 created a chamfer 1006 to provide additional area for accessing the cancellous bone region 1004. The benefit of the chamfer will be apparent when comparing FIG. 14A to FIG. 14B. Of note, in further exemplary embodiments of the present disclosure, cortical cap preservation may be accomplished whereby the tip design preserves the cortical cap so that the surgeon can place the cap back to fill the hole.

With reference to FIG. 14A, a chamfered hole 1006 was drilled into bone 1000 to facilitate the harvesting of cancellous bone 1004. With reference to FIG. 14B, a straight hole 806 was drilled into bone 800 to facilitate the harvesting of cancellous bone 804. In both figures, the width of the cutting tip (not shown) was the same. Device 1100 was designed for bone harvesting and is further discussed in co-pending application, U.S. Publication No. 2015/0045799, assigned to The Johns Hopkins University. Comparing the chamfered hole 1006, FIG. 14A, to the straight hole 806, FIG. 14B, device 1100, which includes shaft 1102 and harvesting tool 1104, can harvest more cancellous bone 1004 from the chamfered hole 1006 as compared to the straight hole 806, when using cutting tips (not shown) of the same width. By including a chamfer, device 1100 can enter the hole at greater angle and access cancellous bone 1004 further away from the vertical axis of the bone 1000. However, both hole configurations have their benefits; as discussed above, the chamfered hole 1006 enables the harvesting of more cancellous bone without requiring a larger cutting tip. Meanwhile, the straight hole 806 can be utilized in areas where there is limited workable service area on the bone 800.

With reference to FIGS. 15A and 15B, an alternative stop mechanism according to the present disclosure is schematically depicted according to the present disclosure. System 1200 includes a pilot hole creator 1201 that includes a handle 1204, a shaft 1206 and a cutting tip 1208 that is configured and dimensioned to penetrate a bone substrate “B”. The features/functions of pilot hole creator 1201 are as described above with other embodiments of the present disclosure. The pilot hole creator 1201 is adapted to cooperate with a cannula 1202 that defines an internal passage that is configured and dimensioned to receive shaft 1206 and cutting tip 1208. However, the diameter of cannula 1202 is selected so as to prevent free passage of handle 1204.

Initially, as pilot hole creator 1201 is introduced to cannula 1202 (as shown in FIG. 15A), at the point that cutting tip 1208 makes contact with bone substrate “B”, gap “D” exists between handle 1204 and cannula 1202. However, as the cutting tip is advanced into the bone substrate “B” (as shown in FIG. 15B), the handle 1504 comes into physical contact with the top surface of cannula 1202, as shown generally by reference number 1210. The physical contact between cannula 1202 and handle 1504 functions as a “stop”, thereby preventing further insertion of cutting tip 1208 into bone substrate “B”. Of note, the physical interaction between cannula 1202 and handle 1504 may take various forms, as will be apparent to persons skilled in the art. Thus, for example, handle 1504 may define extension surface(s) that extend downward from handle 1504 and that engage cannula 1502 to effectuate the stop function. The noted extension surface(s) may be movable relative to handle 1504, thereby allowing adjustment of the depth of penetration permitted by the disclosed stop feature. Gradation indicia may be provided on the extension surface(s) or otherwise as part of the disclosed assembly to permit a user to assess the depth of cutting tip penetration relative to bone substrate “B”. Since the abutting surface 1212 of cannula 1202 is in direct contact with the bone substrate “B”, there is a fixed point of reference for the overall assembly 1200 relative to the bone substrate “B”.

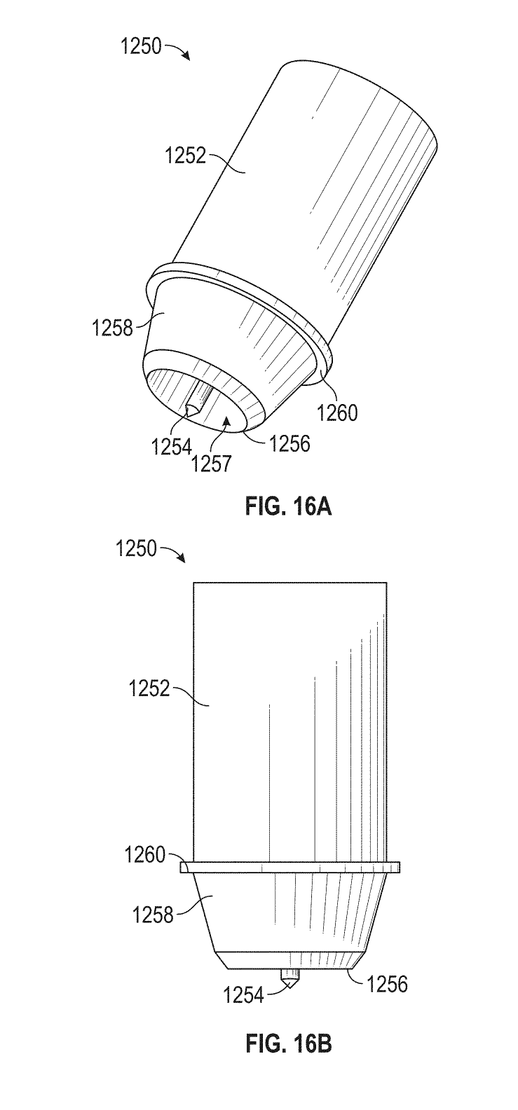

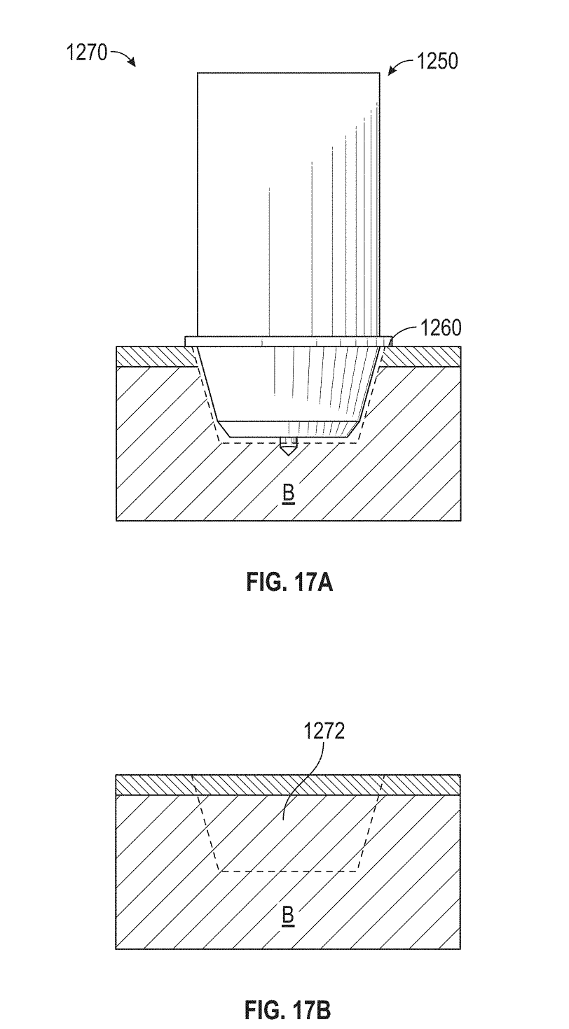

In another exemplary embodiment, with reference to FIGS. 16A and 16B, alternate pilot hole creator 1250 (i.e., tapered cortical cap remover) allows for removal and preservation of a cortical cap, as will be evident from FIGS. 17A and 17B. In a preferred embodiment, tapered cortical cap remover 1250 defines a substantially cylindrical shaft 1252 that abuts stop feature 1260. Although depicted as cylindrical, shaft 1252 is not limited to a cylindrical shape and can be other shapes (e.g., triangular, square, and the like). Extending from stop feature 1260 is a tapered frustoconical feature 1258 with a cutting edge 1256. Although depicted as frustoconical, the tapered feature 1258 can be a variety of shapes, for example, square, cylindrical, rectangular, and the like. Additionally, although depicted as a continuous feature, tapered feature 1258 can be a discontinuous sectioned design having two or more sections (e.g., 2, 3, 4, 5, 6, 7, 8, 9, 10, or more). Axially located along the center axis of tapered cortical cap remover 1250 and extending from stop feature 1260, the center point feature 1254 protrudes a distance past the cutting edge 1256 to ensure first point of contact with the bone. Although depicted with a smooth exterior surface, center point feature 1254 can have additional grip features that enhance fixation with the cortical cap to facilitate removal. Such surface features can include, but are not limited to, dimples, detents, spikes/thorns, and/or a roughened surface.

With reference to the views of FIGS. 16A-17B, the center point feature 1254 of tapered cortical cap remover 1250 engages with the outer surface of bone substrate “B” in the area where bone removal is required. Center point feature 1254 provides a firm connection (i.e., anchor) and a starting point to stabilize and centralize tapered cortical cap remover 1250 as it engages with the bone substrate “B”. As is depicted in assembly 1270, tapered cortical cap remover 1250 can be inserted into bone substrate “B” in a drilling-like motion (i.e., rotational), in a punch-like motion (i.e., translational), or in a combination thereof, wherein cutting edge 1256 cores out a frustoconically shaped piece of bone (i.e., cortical cap) 1272 that recedes into the tapered section 1258 through opening 1257 and can be preserved for reinsertion into the host bone, if necessary.

In some instances, cortical cap 1272 may not be completely cut from the bone substrate “B” based on introduction of the cortical cap remover. Once the tapered cortical cap remover 1250 is fully engaged into the bone substrate “B”, the cortical cap may be broken away from the bone substrate by the user, e.g., by wiggling or torqing the tapered cortical cap remover 1250 so that the cortical cap 1272 severs from bone substrate “B”. In some instances, the cortical cap 1272 will be left in the bone substrate “B” and can be easily removed by the user and, in other instances, the cortical cap 1272 will remain in the tapered section 1258 and with minimal effort can be removed and set aside. A non-smooth center point feature 1254 may be preferable to enhance gripping of the cortical cap 1272 in instances where it is desired/necessary to break the cortical cap away from the bone substrate.

The shape of the cortical cap is defined by the internal shape of the tapered feature 1258. Further, the maximum depth of the hole created in bone substrate “B” is defined by stop feature 1260. In one embodiment, as is depicted in the figures, stop feature 1260 is an integrated continuous circumferentially located feature on shaft 1252. In another embodiment, stop feature 1260 can translate and lock at different heights along the center axis of shaft 1252, thereby customizing the depth of hole as is required.

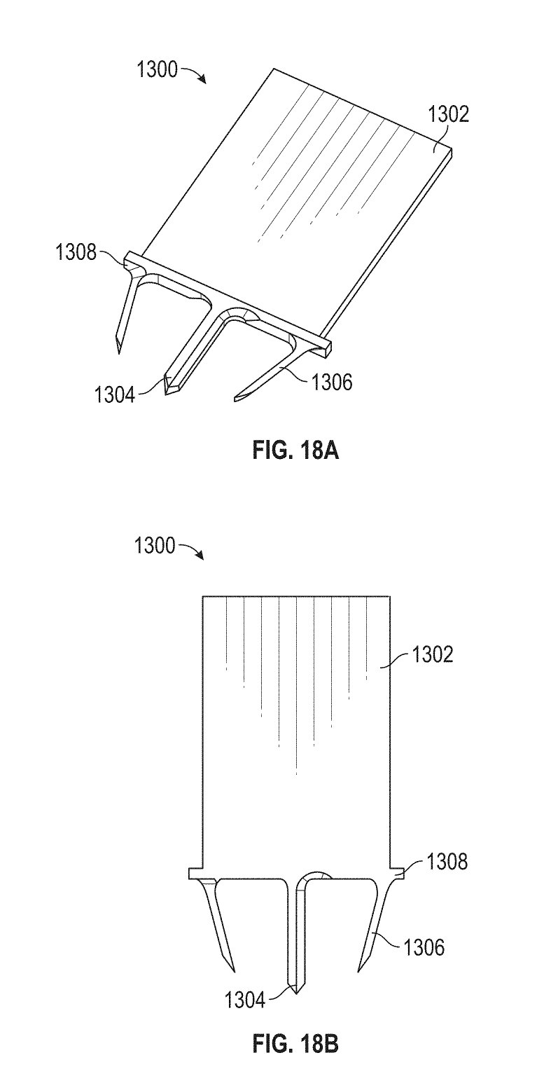

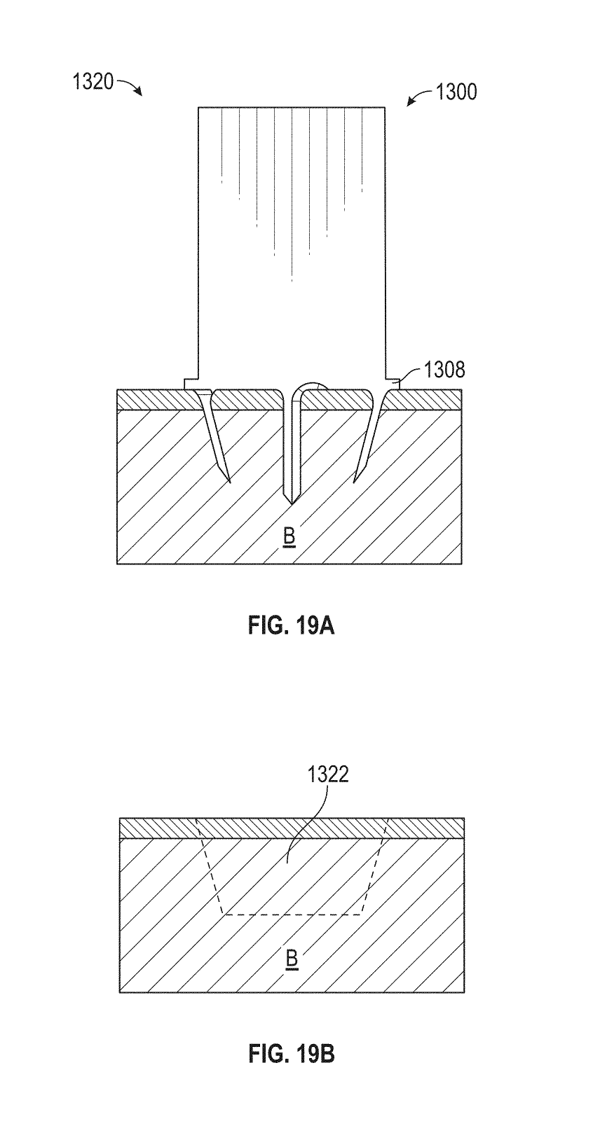

In another exemplary embodiment, with reference to FIGS. 18A and 18B, alternate pilot hole creator 1300 (i.e., compass double point) allows for removal and preservation of a cortical cap, as will be evident from FIGS. 19A and 19B. In a preferred embodiment, compass double point 1300 has a planar attachment feature 1302 that provides connection capabilities to a handle or drill-like device. Although depicted as a planar feature, planar attachment feature 1302 can be a variety of shapes and dimensions, for example, a cylindrical rod, a square rod, and the like. At the base of planar attachment feature 1302 (closest to the cutting flutes) is stop feature 1308, which as described above, functions to limit the depth of cut. Extending from stop feature 1308 is a centrally located axial center point feature 1304 that extends a distance past the cutting flutes 1306 to ensure first point of contact with the bone. Although depicted with a smooth exterior surface, center point feature 1304 can have additional grip features that enhance fixation with the cortical cap to facilitate removal, such features can include, but are not limited to dimples, detents, spikes/thorns, and/or a roughened surface. In a preferred embodiment, two cutting flutes 1306 are tapered inward to produce a frustoconically shaped piece of bone. In another embodiment, more than two (e.g., 3, 4, 5, 6 or more) cutting flutes can be utilized without separating from the intent of the disclosure.

With reference to the views of FIGS. 18A-19B, the center point feature 1304 of compass double point 1300 engages with the outer surface of bone substrate “B” in the area where bone removal is required. Center point feature 1304 provides a firm connection (i.e., anchor) and a starting point to stabilize and centralize compass double point 1300 as it engages with the bone substrate “B”. As is depicted in assembly 1320, compass double point 1300 can be inserted into bone substrate “B” in a drilling-like motion (i.e., rotational), wherein cutting flutes 1306 core out a frustoconically shaped piece of bone (i.e., cortical cap) 1322 that can be preserved for reinsertion into the host bone, if desired and/or necessary. The drilling-like motion can be accomplished by a hand tool or a drill-like device (i.e., manual or device/power assisted). In some instances, cortical cap 1322 may not be completely cut from the bone substrate “B”, in such cases, a non-smooth center point feature 1304 is preferable to grip the cortical cap 1322. In those instances, once the compass double point 1300 is fully engaged into the bone substrate “B”, the user may wiggle/torque the compass double point 1300 so that the cortical cap 1322 severs from bone substrate “B”. In some instances, the cortical cap 1322 will be left in the bone substrate “B” and can be easily removed by the user, and in other instances, the cortical cap 1322 will remain in between the cutting flutes 1306 and with minimal effort can be removed and set aside.

The shape of the cortical cap is generally defined by the angle of the cutting flutes 1306 and can be anywhere from substantially cylindrical to substantially conical, or any angle in between. Further, the maximum depth of the hole created in bone substrate “B” is defined by stop feature 1308. In one embodiment, as is depicted in the figures, stop feature 1308 is an integrated protrusion extending from either side of planar attachment feature 1302. In another embodiment, stop feature 1308 can translate and lock at different heights along the vertical center axis of planar attachment feature 1302, thereby customizing the depth of hole as is desired and/or required.

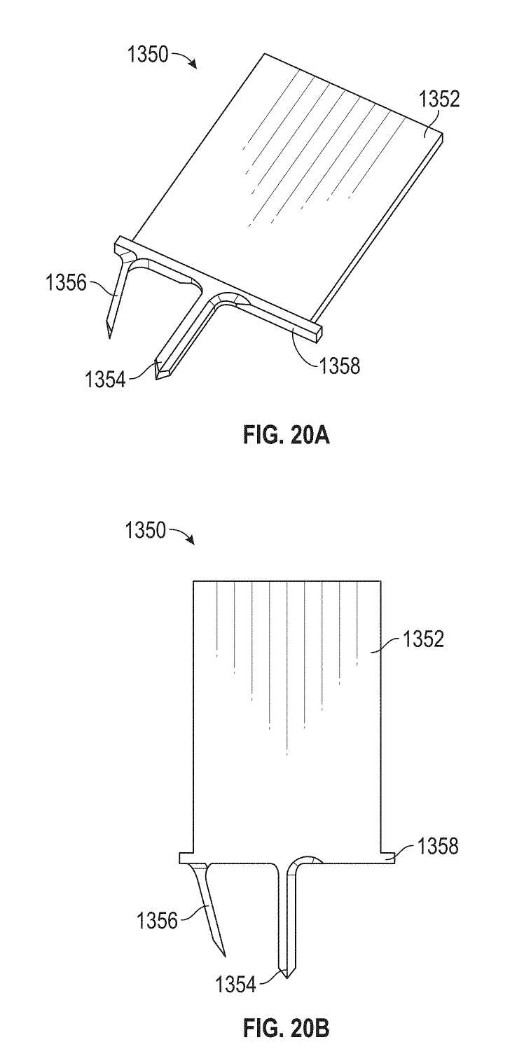

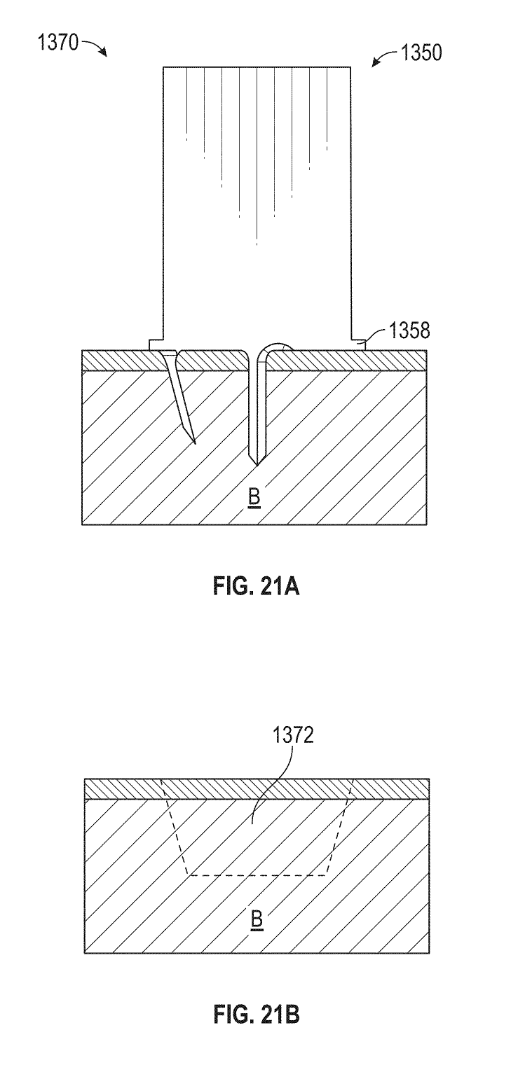

In another exemplary embodiment, with reference to FIGS. 20A and 20B, alternate pilot hole creator 1350 (i.e., compass single point) allows for removal and preservation of a cortical cap, as will be evident from FIGS. 21A and 21B. In a preferred embodiment, compass single point 1350 has a planar attachment feature 1352 that provides connection capabilities to a handle or drill-like device. Although depicted as a planar feature, planar attachment feature 1352 can be a variety of shapes and dimensions, for example, a cylindrical rod, a square rod, and the like. At the base of planar attachment feature 1352 (closest to the cutting flute) is stop feature 1358, which as described above, functions to limit the depth of cut. Extending from stop feature 1358 is a centrally located axial center point feature 1354 that extends a distance past the cutting flute 1356 to ensure first point of contact with the bone. Although depicted with a smooth exterior surface, center point feature 1354 can have additional grip features that enhance fixation with the cortical cap to facilitate removal, such features can include, but are not limited to dimples, detents, spikes/thorns, and/or a roughened surface. Cutting flute 1356 is tapered inward to produce a frustoconically shaped piece of bone.

In view of FIGS. 20A-21B, the center point feature 1354 of compass single point 1350 engages with the outer surface of bone substrate “B” in the area where bone removal is required. Center point feature 1354 provides a firm connection (i.e., anchor) and a starting point to stabilize and centralize compass double point 1300 as it engages with the bone substrate “B”. As is depicted in assembly 1370, compass single point 1350 can be inserted into bone substrate “B” in a drilling-like motion (i.e., rotational), wherein cutting flute 1356 cores out a frustoconically shaped piece of bone (i.e., cortical cap) 1372 that can be preserved for reinsertion into the host bone, if desired and/or necessary. The drilling-like motion can be accomplished by a hand tool or a drill-like device (i.e., manual or device/power assisted). In some instances, cortical cap 1372 may not be completely cut from the bone substrate “B”, in such cases, a non-smooth center point feature 1354 is preferable to grip the cortical cap 1372. In those instances, once the compass single point 1350 is fully engaged into the bone substrate “B”, the user may wiggle/torque the compass single point 1350 so that the cortical cap 1372 severs from bone substrate “B”. In some instances, the cortical cap 1372 will be left in the bone substrate “B” and can be easily removed by the user and, in other instances, the cortical cap 1372 will remain attached between the center point feature 1354 and the cutting flute 1356 and with minimal effort can be removed and set aside.

The shape of the cortical cap is defined by the angle of cutting flute 1356 and can be anywhere from substantially cylindrical to substantially conical, or any angle in between. Further, the depth of the hole created in bone substrate “B” is defined by stop feature 1358. In one embodiment, as is depicted in the figures, stop feature 1358 is an integrated protrusion extending from either side of planar attachment feature 1352. In another embodiment, stop feature 1358 can translate and lock at different heights along the vertical center axis of planar attachment feature 1352, thereby customizing the depth of hole as is required.

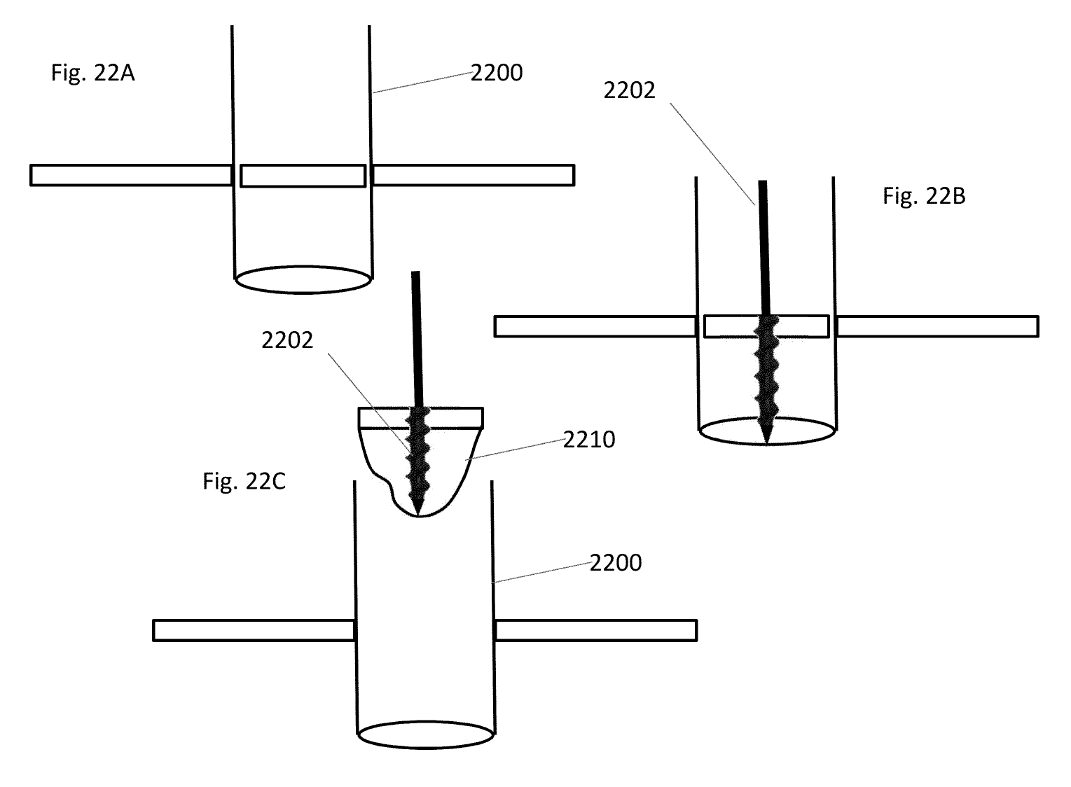

In another exemplary embodiment, FIGS. 22A-22C depict an alternative pilot hole creator for removing and preserving a cortical cap. FIG. 22A depicts a cylindrical instrument 2200 that may be inserted axially through a cortical layer and into a cancellous region of bone. FIG. 22B depicts a screw 2202 that may be inserted concentrically through the cylindrical instrument 2200 and screwed through the cortical and cancellous regions of bone. FIG. 22C depicts the removal of the inner screw 2202 with a cortico-cancellous plug 2210 detached from the rest of the boney region. In an exemplary embodiment, the cylindrical instrument 2200 and screw 2202 may be inserted and removed simultaneously. In an exemplary embodiment, the cylindrical instrument 2200 and screw 2202 may be inserted and removed in series. Screw 2202 may be operable with hand tool or a drill-like device (i.e., manual or device/power assisted).

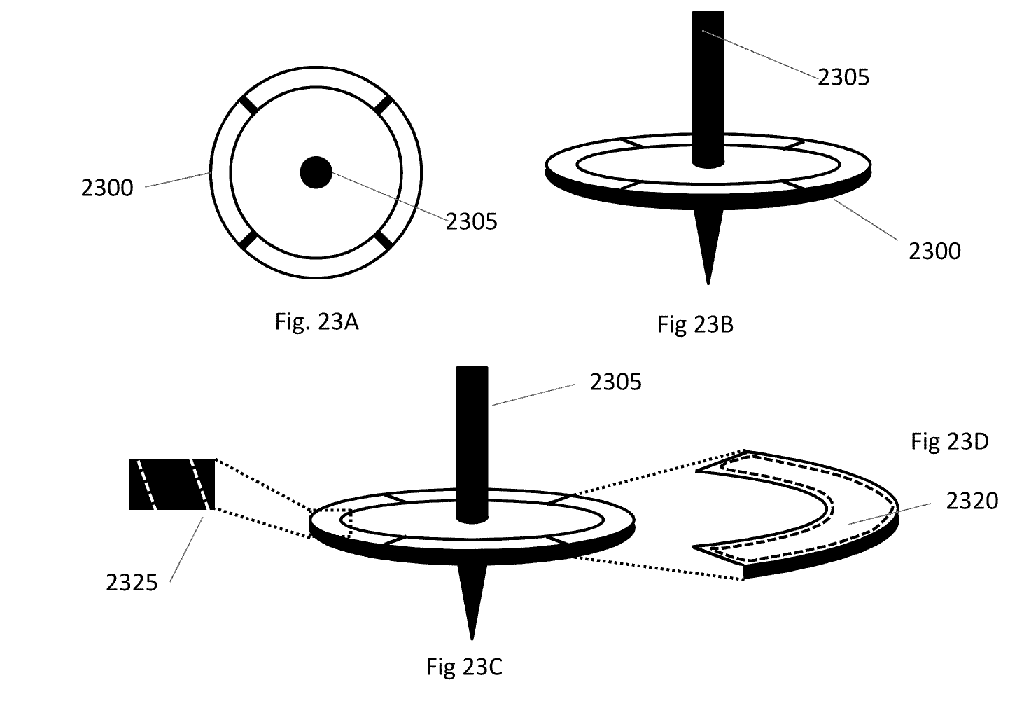

In another exemplary embodiment, FIGS. 23A-23D depict an alternative pilot hole creator embodiment for removing and preserving a cortical cap. FIG. 23A depicts a top perspective view of a guide tool or system 2300 with four quadrants and a central pin 2305. FIG. 23B depicts a front elevational view of the guide system 2300. The central pin 2305 may be embedded into a cortical layer of bone. Each quadrant of the guide system 2300 may contain a slot 2320 that creates an angled channel 2325 through the guide system 2300. In an exemplary embodiment, the angled channel 2325 may extend through a portion or the entirety of the guide system 2300. FIG. 23C depicts a cross-sectional view of an exterior-most portion of the guide system 2300 and traces the angled channel 2325. FIG. 23D depicts an enlarged, perspective view of one quadrant of the guide system 2300 and depicts the slot 2320. While this embodiment of the guide system 2300 contains four quadrants, any number of slotted regions and/or slots may be used. For example, each quadrant of the four quadrants may contain one slotted region or multiple slotted regions. Each quadrant may also contain a different number of slotted regions that the remaining quadrants.

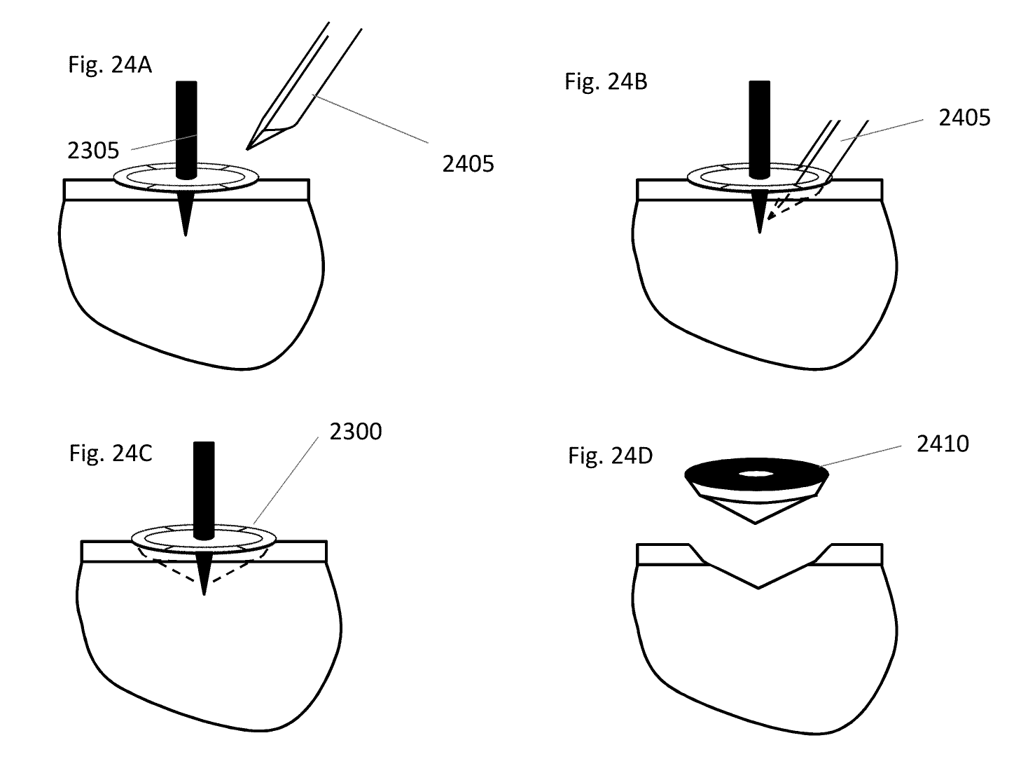

FIGS. 24A-24D depict the guide system 2300, as shown in FIGS. 23A-23D, during an exemplary use case. In some embodiments, the guide system 2300 may be used to remove a cortical cap. FIG. 24A shows the guide system 2300 fixed to a cortical surface by insertion of the central pin 2305 through the cortical layer and into the cancellous region of bone. FIG. 24A further illustrates a punch tool 2405 that may be formed to fit the slot 2320 on one of the quadrants on the exterior surface of the guide system 2300. FIG. 24B depicts insertion of the punch tool 2405 through one of the slots 2320, through the cortical layer, and into the cancellous region of bone. FIG. 24C depicts a conical outline of the region cut by the punch tool 2405 once the tool 2405 is inserted through each of the four quadrants of the guide system 2300. FIG. 24D shows a resulting cortical cap 2410 that is removed from the native boney region. Cortical cap 2410 may be extracted from the native boney region according to aspects of the present disclosure taught herein.

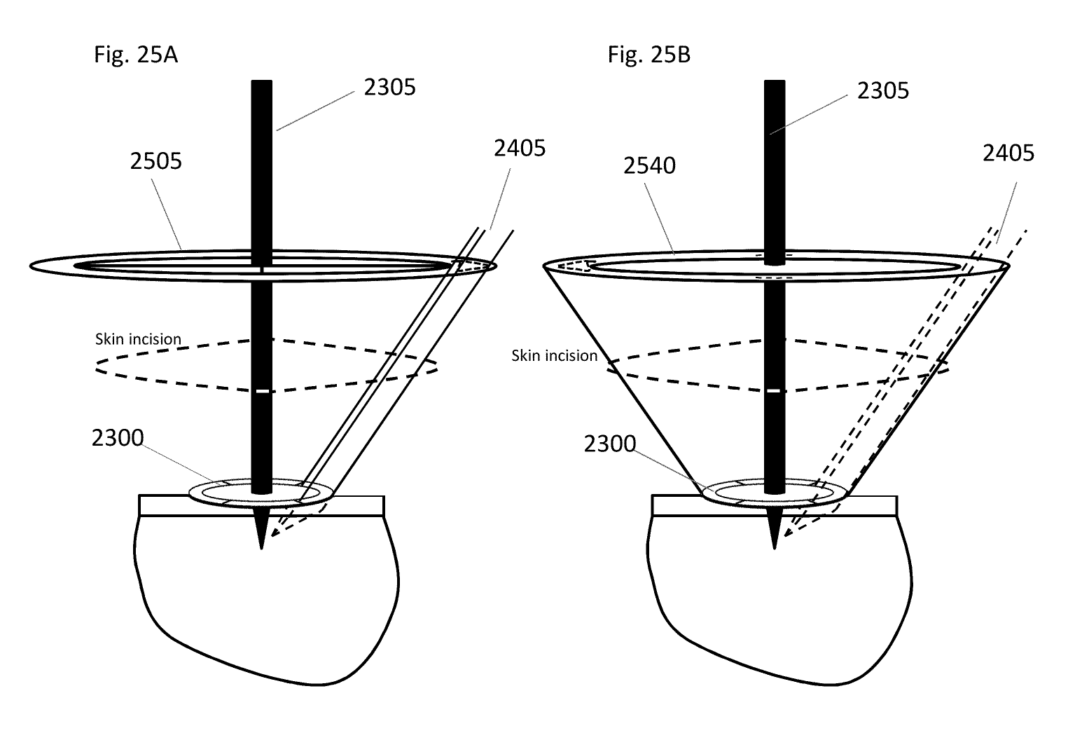

FIGS. 25A-25B depicts two embodiments of ancillary features of the guide system 2300 that may be used to assist a user (e.g., physician, surgeon, or other suitable profession) in aligning the punch tool 2405 with each slot 2320 of the guide system 2300. This assisted guidance may be necessary because the guide system 2300 may be used or employed several centimeters below the skin incision line in clinical practice, providing a small window of visibility through which to align the punch tool 2405 into the slots 2320 of the guide system 2300. FIG. 25A depicts a second guide ring 2505 attached to the central pin 2305 above the skin incision with slots sized to accept the punch tool 2405. The second guide ring 2505 may be positioned at a desired height and diameter such that when the punch tool 2405 is inserted through one of the slots 2320, the punch tool 2405 is forced into alignment with the slot 2320 of the guide tool 2300, which is fixed to the cortical surface of the bone. In an exemplary embodiment, the placement and diameter of the second guide ring 2505 may be adjustable to change the angle of entry of the punch tool 2405 into the native boney region. FIG. 25B depicts an alternative guidance tool 2540 having a conical shape with pre-cut channels that may be configured to accept the insertion of the punch tool 2405 outside of the skin incision and guide the punch tool 2405 into each respective slot 2320 of the guide system 2300 atop the cortical bone.

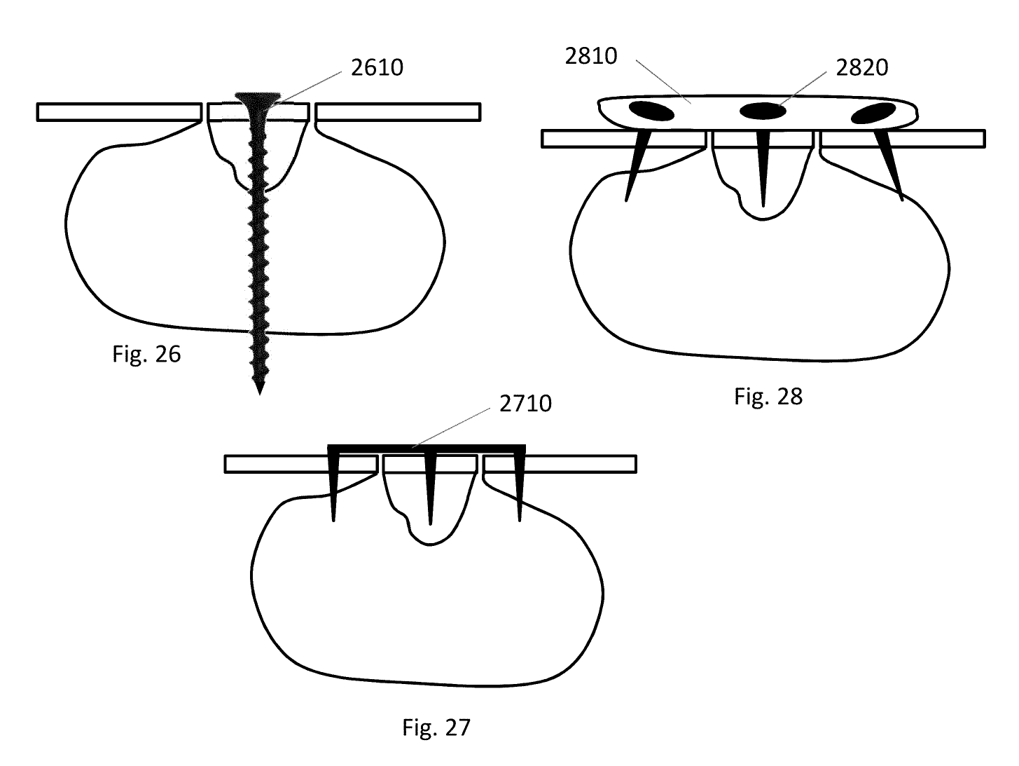

In another exemplary embodiment, FIGS. 26-28 show various embodiments of fastening a cortical cap back into a cortical hole, according to aspects of the present disclosure taught herein. Each figure depicts a cortical layer of bone over a cancellous region of bone that has been harvested, leaving behind a void in the boney region. FIG. 26 depicts a screw 2610 configured to be inserted through a cortical cap. Screw 2610 may be configured to be screwed into a contralateral side of a boney void, as depicted. The contralateral side may be comprised of cancellous bone, cortico-cancellous bone, or cortical bone. Turning to FIG. 27, shown is a three-pronged staple 2710 that may be configured to be fastened to the cortical cap within the cortical hole. One prong of the staple 2710 may be fixed to the cortical cap, while the remaining two prongs of the staple 2710 may be anchored to the intact cortical layer on either side of the cortical hole. The prongs, as depicted, may be perpendicularly formed to the spine of the staple 2710. In some embodiments, the prongs of the staple 2710 may be angled to various degrees. While the embodiment depicted in FIG. 27 depicts three prongs, it can be appreciated that other embodiments may comprise a greater or fewer number of prongs. FIG. 28 depicts a plate 2810 and pin 2820 construct that may be used to fasten a cortical cap within the cortical hole. The plate 2810 may extend or span over the cortical hole and may contain three holes through which pins 2820 (or screws) may be passed at variable or pre-set angles. The two outer most located pins 2820, which are shown in FIG. 28 may be anchored on each side of the cortical hole, while the central pin 2820 may be used to fix or secure the cortical cap in place. The plate 2810 may be shaped in various configurations. It can be appreciated that the number and orientation of pins and/or screw holes may vary in different embodiments than from the embodiment shown in FIG. 28.

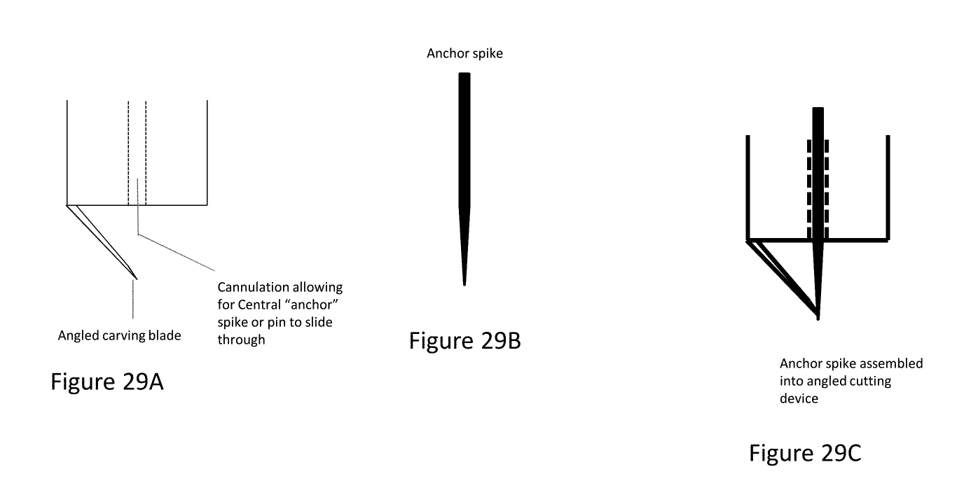

In another exemplary embodiment, and according to aspects taught by the present disclosure herein, FIGS. 29A-29C show an exemplary angled carver device, or angled cutting device, with a central retractable anchor. FIG. 29A depicts the angled carver with an angled carving blade. The angled carver may include cannulation, which may allow for a central anchor spike or anchoring pin to slide therethrough. An exemplary anchor spike is shown in FIG. 29B. FIG. 29C depicts the anchor spike in a fully assembled form within the angled carver device.

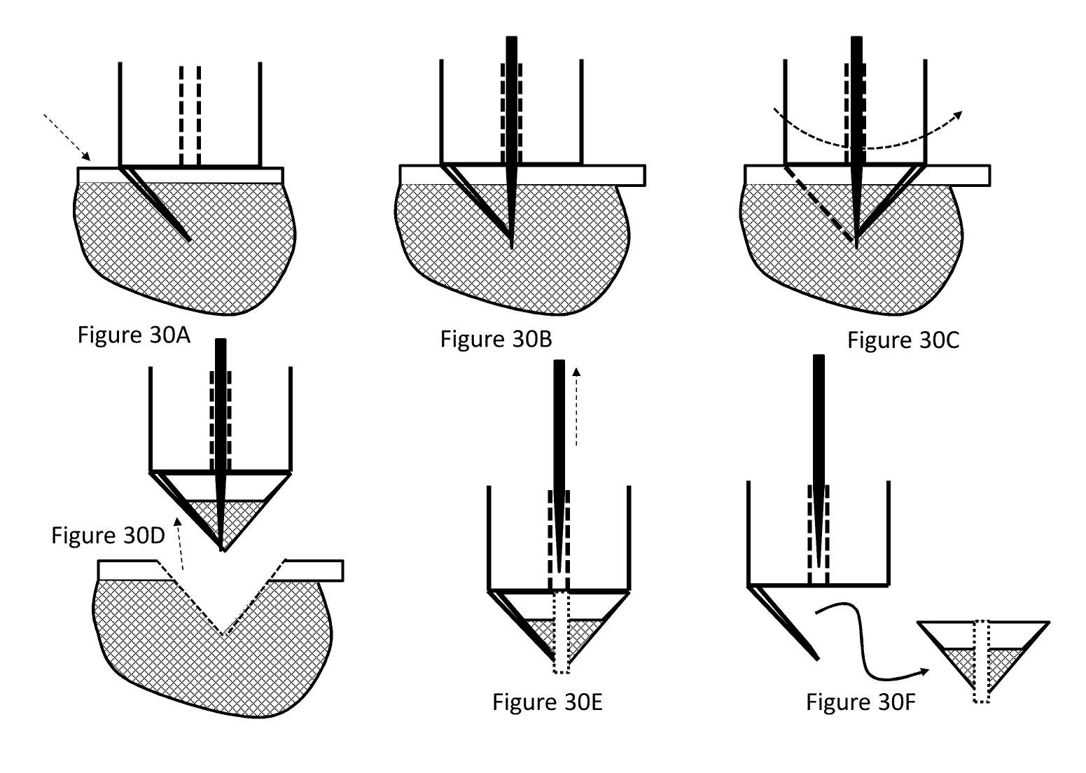

FIGS. 30A-30F depict an exemplary use case of the angled carver depicted and described with respect to FIGS. 29A-29C. As shown in FIG. 30A, the angled carver may be inserted at an angle relative to a cortical layer of bone. The anchor spike may be passed or inserted in, as shown in FIG. 30B, to secure the device. The anchor spike may be secured by being pushed, hammered, threaded, or screwed into the adjacent bone. The angled carver may be configured to rotate centrally or concentrically relative to the central anchor or central spike. The angled carver may be actuated (FIG. 30C) clockwise or counterclockwise, or a combination of both, until the cortical cap or plug is retrieved. FIG. 30D shows an exemplary use of the carver where the cortical plug is retrieved. FIG. 30E shows the central anchor being removed from the cortical plug. FIG. 30F shows how the cortical plug is obtained from the angled carver. It can be appreciated that the cortical plug or cap can be obtained, retrieved, or otherwise manipulated, according to aspects of the present disclosure taught herein.

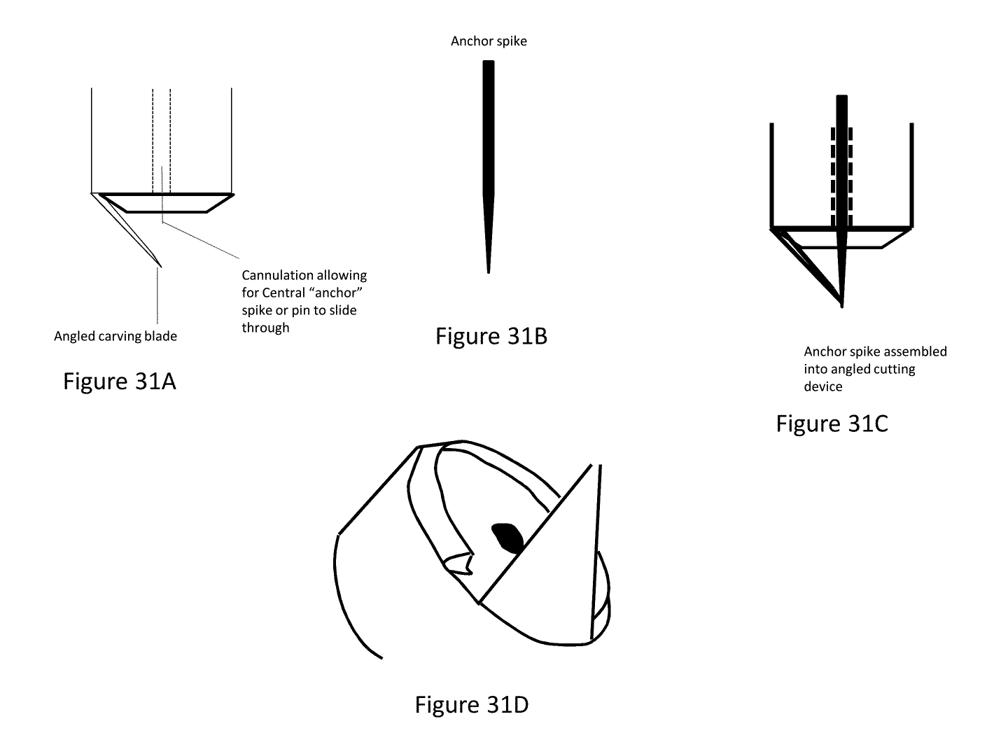

In another exemplary embodiment, and according to aspects taught by the present disclosure herein, FIGS. 31A-31D show another exemplary angled carver device, or angled cutting device, comprising a central retractable anchor and a ring blade. FIG. 31A shows the angled carving blade of the angled carver device. Cannulation within the device may allow a central anchor spike or pin to be slideably insertable therethrough. FIG. 31A also depicts the ring blade, which upon piercing the cortex of bone, may create a stencil or outline for the carving blade to follow as it rotates through the cutting path created by the device. FIG. 31B depicts an exemplary anchor spike that may be used with the angled carver. FIG. 31C depicts the anchor spike assembled into the angled carver device. In FIG. 31D, shown as isometric view, the ring blade may be continuous or may be interrupted to form partitioned angled “teeth” that may pierce the cortex. Upon subsequent rotation of the angled blade of the angled carver about the anchor spike, the ring blade may pre-fabricate a cut line for the angled carver blade to be rotated through and cut the shape more easily. This is similar to a pre-cut drill hole to ease the cutting of a drill pass.

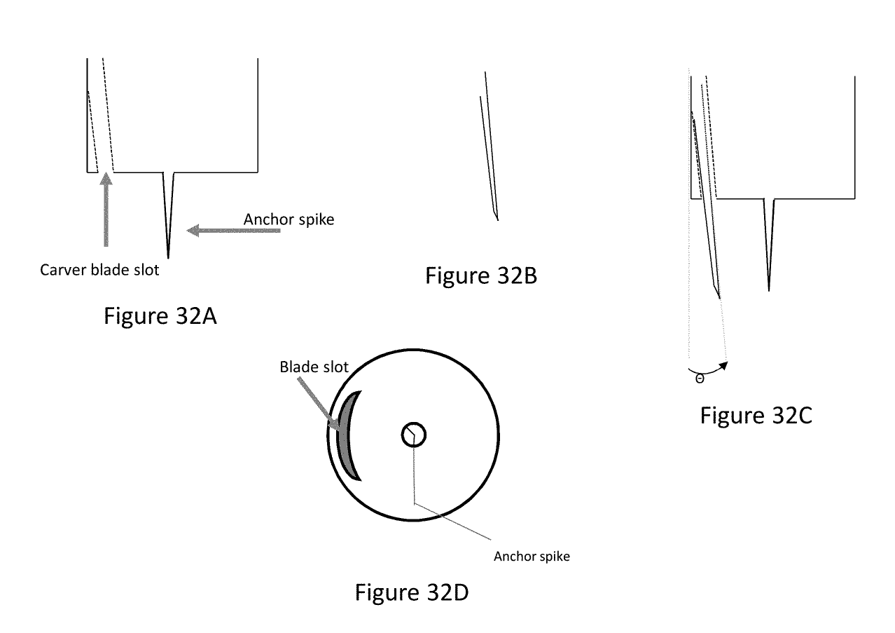

FIGS. 32A-32-D further describe an depict the angled carver of FIGS. 31A-31D. The angled carver may comprise a carver blade slot and the anchor spike, as shown in FIG. 32A. With respect to this embodiment of the carver blade, and with reference to FIG. 32B, the blade may flexible such that it may enter the carver blade slot from a proximal end of the device. Upon sliding through a distal end of the blade slot, a deflection angle may be introduced. As the carver blade is axially loaded from the proximal end of the handle, the carver blade may pierce the bone surface at an angle Θ, as shown in FIG. 32C. In this embodiment of the angled carver, the carver blade may comprise cutting surfaces that may enable piercing through an axial load and cutting or carving through rotation of the blade relative to the central anchor spike. FIG. 32D depicts a top view of the distal end of the angled carver and illustrates the blade slot and the anchor spike.

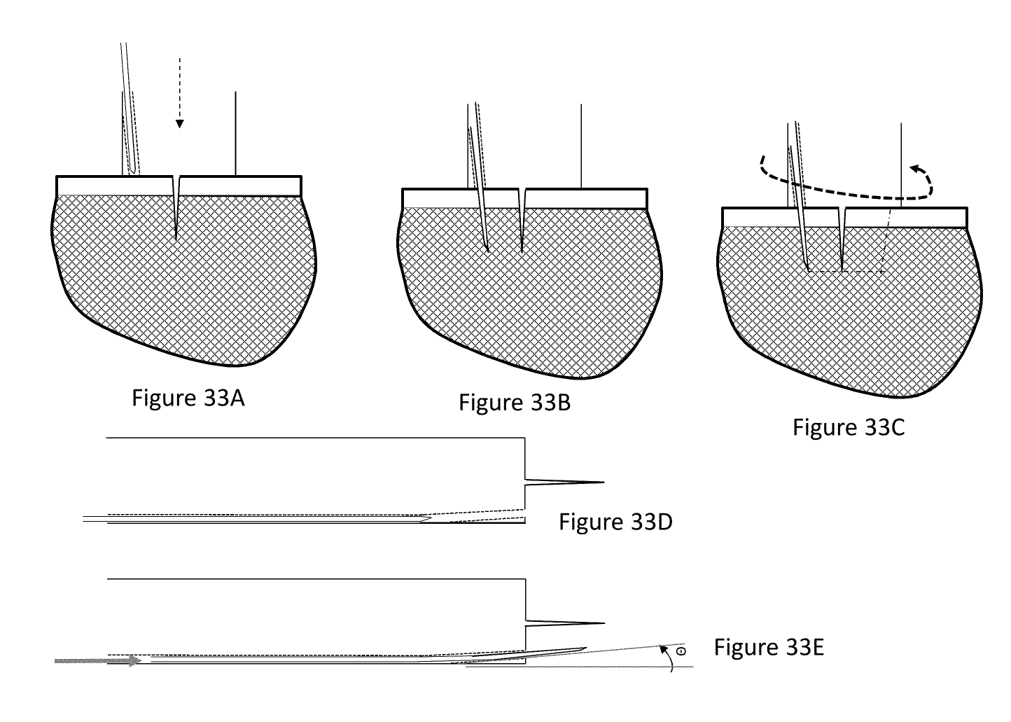

FIGS. 33A-33E depict the angled carver taught herein further comprising a retractable carving blade. In this embodiment, retrieval of a plug may be performed according to aspects of the present disclosure as taught herein. As shown in FIG. 33C, once the plug is retrieved, the angled blade may be retracted, instead of the retractable central anchor as described and depicted in earlier embodiments. The angled carver may deflect into a piercing angle, through axial translation, as shown in FIGS. 33D-33E.

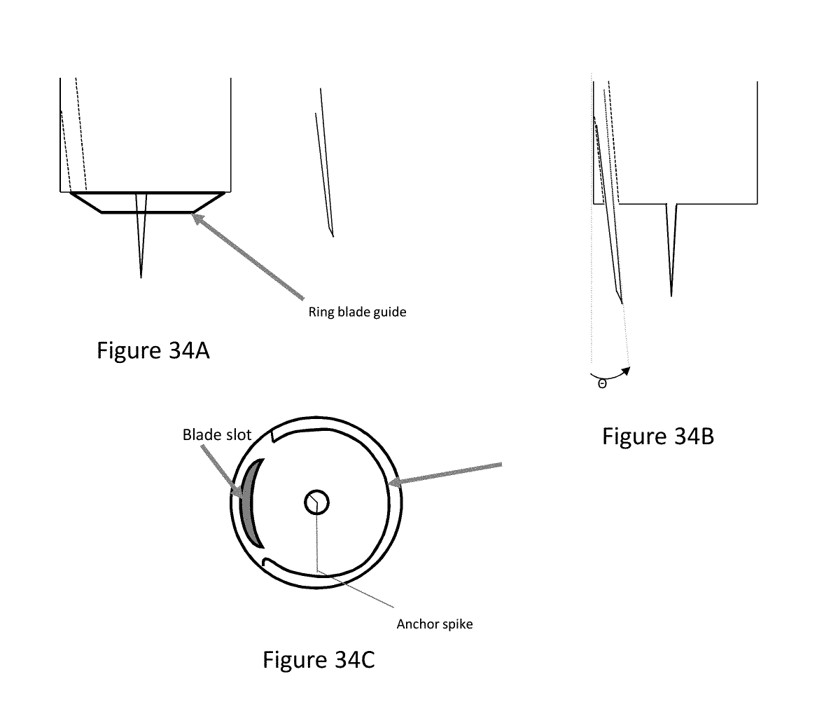

In another exemplary embodiment, and according to aspects taught by the present disclosure herein, FIGS. 34A-34C depict and describe an additional embodiment of an angled carver comprising a retractable carving blade and a ring blade. FIG. 34A depicts the ring blade and a channel for the angled carver. The ring blade and the angled carver may be configured such that the ring blade may be capable of pre-cutting a circle for the angled carver to follow through as a stencil so as to reduce the cutting force necessary to rotate the angled carver.

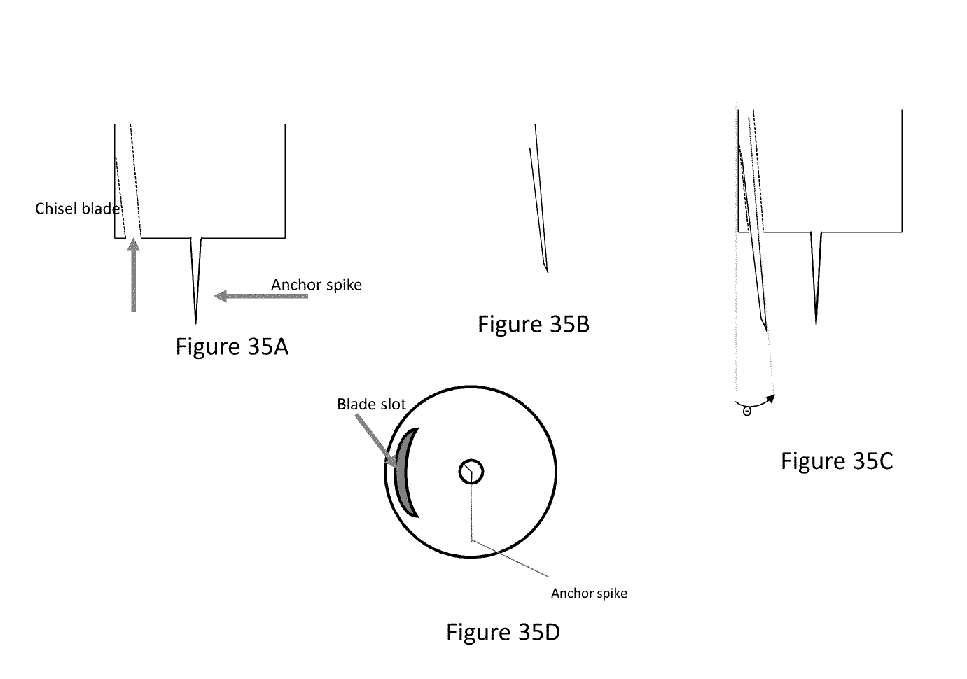

In another exemplary embodiment, and according to aspects taught by the present disclosure herein, FIGS. 35A-35D depict a chisel punch blade with a downward cutter. In this embodiment, the cutting of the cortical plug may be created in series. During operation of the chisel punch blade, a user may pierce the cortex with an anchor spike, as taught herein, and axially translate the chisel blade so that it pierces the bone. Once the bone is pierced, the chisel blade may be retracted, and the device may be rotated by a desired increment such that the chisel punch blade may pierce into uncut bone. This may be repeated until a full conical shape has been cut out by the punch blade. This may be further combined with the ring blade, as taught herein, to reduce the piercing force of the blade to deflect through as it is axially translated into the bone. This embodiment may further include a ring blade to secure the device as well as provide a stencil cut for the chisel punch to reduce the cutting or piercing force necessary.

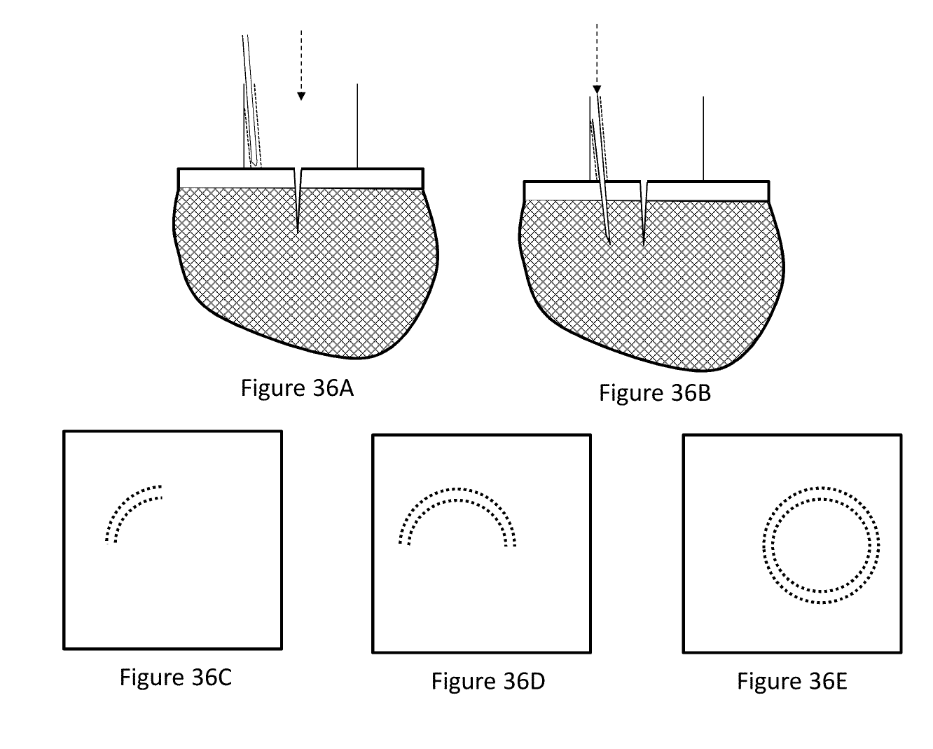

FIGS. 36A-36E depict exemplary operation of the chisel punch blade of FIGS. 35A-35D. In FIG. 36C, showing a top view of the bone surface, a first “chisel punch” is shown. A user may then retract the chisel punch blade, rotate the device about the central anchor (or spike), and repeat a “chisel punch” to create another cut, as shown in FIG. 36D. This process may be continually performed by the user until a full cortical plug is cut from the boney surface, as shown in FIG. 36E.

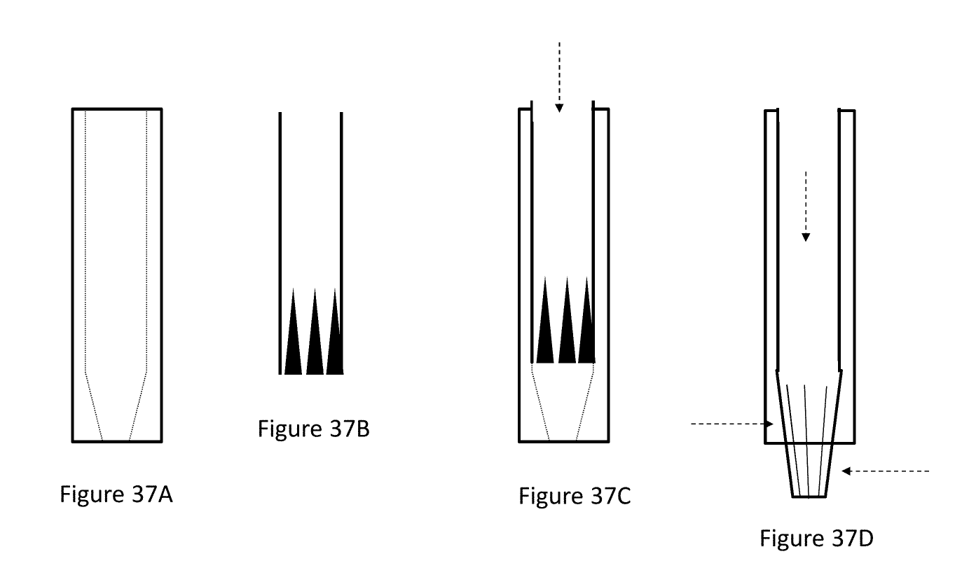

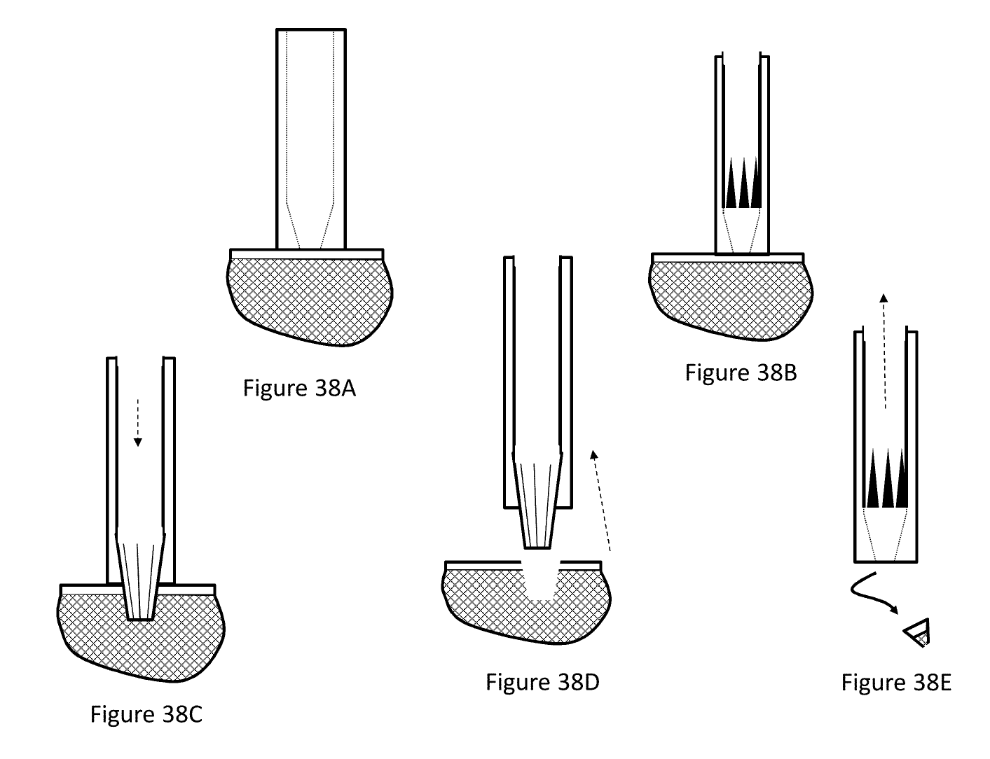

FIGS. 37A-37D depict another exemplary embodiment of a cutting device according to aspects of the present disclosure. A multi-slotted blade expandable “flower” is depicted by the figures. FIG. 37A depicts the device's outer guide sheath. FIG. 37B depicts the multi-blade punch/cutter with deflectable blades. The multi-blade punch/cutter may be threaded through the guide sheath, as shown in FIG. 37C. Finally, as shown in FIG. 37D, the multi-blade punch may pass through a deflection area of the outer guide sheath, which may cause the blades to compress together. In this embodiment, the cutting device may comprise one or more chisel blades that may pierce upon axially translating the blade element that contains the deflectable piercing chisel blades. The cutting device may also be configured such that the number of punch blades can be completed or combine together to form a full circumference necessary to remove a cortical plug after one full axial translation of the chisel blades. The cutting blades may flex into a conical shape upon translating through the deflection area of the outer sheath. FIGS. 38A-38E depict an exemplary operation of the multi-slotted blade of FIGS. 37A-37D to retrieve a cortical plug, according to aspects of the disclosure taught herein.

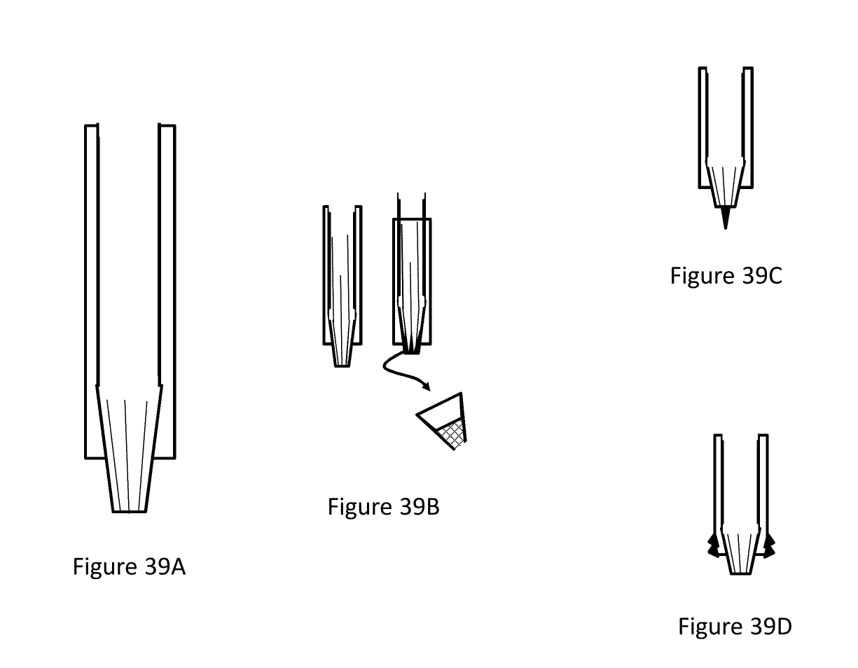

The multi-slotted blade of the embodiments described and depicted herein (FIGS. 37A-37D) may further comprise a central cannulation, as shown in FIGS. 39A-39D. In this embodiment, the central cannulation may be configured to accept an anchor spike to pass through so that the cutting can be further guided (FIG. 39C). The outer sheath may include an anchor feature such that the anchor feature may be first secured onto the cortical surface prior to actuating the multi blade element through the deflection guide (FIG. 39D). This may be in the form of an outer thread that may fasten the outer sheath to the cortex in order to stabilize the outer sheath when operating the multi-slotted blade. The multi-slotted blade cutter may also be configured to cut via axial hammering through a piercing cut. Alternatively, the multi-slotted blade cutter may be configured to be rotated while simultaneously implementing an axial translation so that the blades may carve via rotation while the device is piercing. This may be graduated through the use of an inner thread or rail mechanism. In an exemplary embodiment, the multi-slotted blade cutter may comprise slots of a calibrated length such that after obtaining the plug, as taught herein, and as the multi blade cutter is retracted and the blade teeth begin separating, the multi-slotted blade cutter enables the core plug to be released as the space in between the teeth increase as it is retracted from the outer sheath once the plug is obtained (FIG. 39B). In yet another exemplary embodiment, the multi-slotted blade cutter may be removed from the outer sheath while maintaining a grip on the cortical plug. Once removed from the outer guide sheath, a plunger may be used to pop the cortical plug out of the embodiment as the blades are in their expanded position.

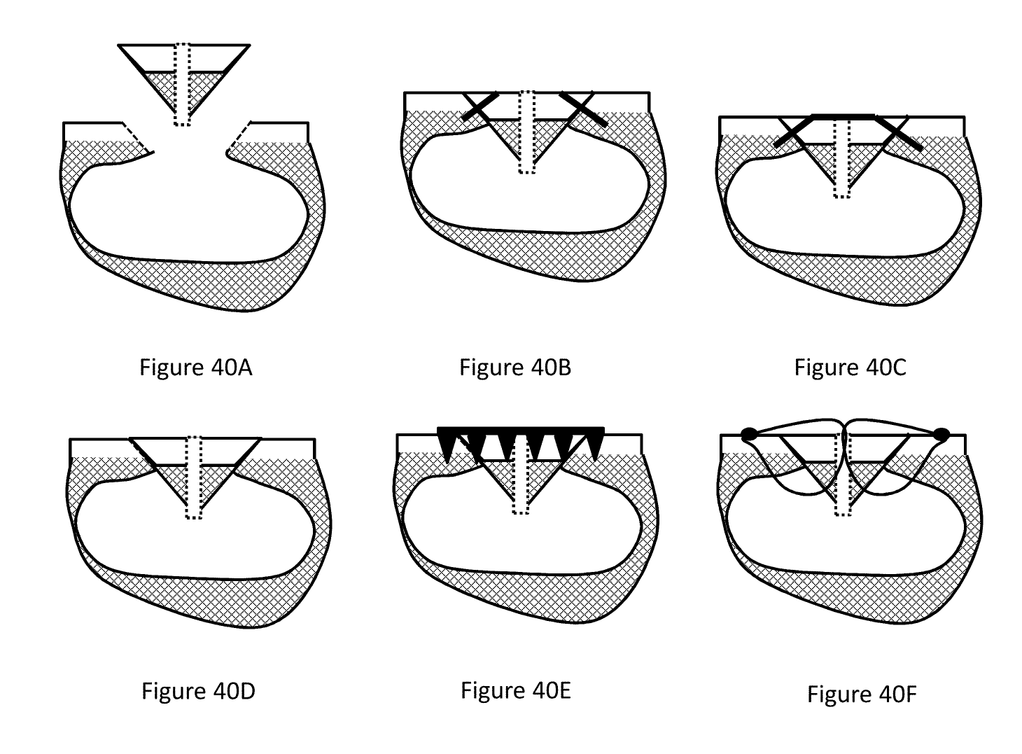

FIGS. 40A-40F depict additional concepts for fastening, according to aspects of the present disclosure taught herein. For example, FIG. 40B depicts fastening using a pin or screws. FIG. 40C depicts fastening using a staple. A rasped plate, as shown in FIG. 40E, may be used for fastening. Lastly, sutures may also be used for fastening, as shown in FIG. 40F.

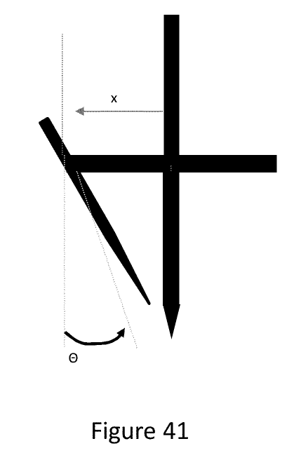

With respect to FIG. 41, an adjustable hole saw is depicted. In this embodiment, the hole saw may be configured to include an adjustable diameter. The adjustable diameter may be adjusted by translating the cutting blade from a central axis by a distance x, as shown in FIG. 41. Furthermore, the blade may also be configured to achieve angular rotation if a tapered cut is desired using angle Θ, as depicted. In such an embodiment, one or more cutting blades may be provided. This may enable adjustability for plug size to accommodate various anatomies and size requirements for the user.

Although the present disclosure has been described with reference to exemplary embodiments and implementations thereof, the present disclosure is not limited by or to such exemplary embodiments/implementations. Rather, the devices, systems and methods disclosed herein may be modified, enhanced and/or refined without departing from the spirit or scope of the present disclosure.

1. A bone-cutting device, comprising:
a. a cutting tip configured to penetrate bone; and
b. a holder adapted to be associated with the cutting tip;
wherein the cutting tip includes (i) a first portion that functions to anchor the cutting tip relative to a bone substrate, and (ii) one or more flutes that function to remove bone fragments from the bone substrate;
wherein the bone cutting device defines a stop feature to control the depth of insertion of the cutting tip relative to the bone substrate.

2. The bone-cutting device of claim 1, wherein the holder defines a shaft and wherein the association between the holder and the cutting tip is selected from the group consisting of (i) a fixed connection between the shaft and the cutting tip, (ii) a detachable connection between the shaft and the cutting tip, and (iii) integral formation of the shaft and the cutting tip.

3. The bone-cutting device of claim 2, wherein the shaft includes gradation marks.

4. The bone-cutting device of claim 2, wherein the cross-section of the shaft is greater than the width of the cutting tip, and wherein the increased cross-section of the shaft relative to the cutting tip functions as the stop feature.

5. The bone-cutting device of claim 2, wherein the shaft is associated with a handle.

6. The bone-cutting device of claim 5, wherein the handle is a manual handle.

7. The bone-cutting device of claim 5, wherein the handle is movable between a perpendicular orientation relative to the shaft and an axial orientation relative to the shaft.

8. The bone-cutting device of claim 1, wherein the holder includes a sliding feature that is adapted to move axially relative to a shaft associated with the holder, the sliding feature functioning (i) to encapsulate the cutting tip during storage/transport, and (ii) to move into a handle position during use.

9. The bone-cutting device of claim 8, wherein the handle is adapted to rotate and lock from an axial position to a perpendicular position, both relative to the shaft.

10. The bone-cutting device of claim 2, wherein the shaft is adapted to be inserted into a powered drill.

11. The bone-cutting device of claim 1, wherein the cutting tip includes an axially positioned triangular cutting feature that extends therefrom.

12. The bone-cutting device of claim 1, wherein the cutting tip defines surfaces that are radiused to reduce the stress applied to the cutting tip.

13. The bone-cutting device of claim 1, wherein the cutting tip is removable and/or detachable from the holder.

14. The bone-cutting device of claim 1, wherein the cutting tip defines six cutting surfaces.

15. The bone-cutting device of claim 1, wherein the cutting tip defines two flutes.

16. The bone-cutting device of claim 1, wherein the cutting tip defines a substantially flat bone interfacing cutting surface.

17. The bone-cutting device of claim 1, wherein the cutting tip is adapted to create a substantially straight hole.

18. The bone-cutting device of claim 1, wherein the cutting tip is adapted to create a chamfer that leads into a straight hole.

19. The bone-cutting device of claim 1, further comprising a cannula that defines a passage that is configured and dimensioned to receive the cutting tip and a portion of the holder, and wherein interaction between a handle associated with the holder and the cannula functions as the stop feature.

20. A method of creating a pilot hole in bone, comprising:
a. providing a bone-cutting device according to any of the preceding claims, and
b. penetrating a bone substrate with the cutting tip of the bone-cutting device; and
c. engaging a stop feature associated with the bone-cutting device to limit further penetration of the bone-cutting device.

21. A cortical cap removal device, comprising:
a. a shaft;
b. a cutting feature associated with the distal end of the shaft, the cutting feature including (i) a center point feature, and (ii) a cutting element that surrounds, at least in part, the center point feature and that is configured and dimensioned to cut a cortical cap from a bone substrate.

22. The cortical cap removal device according to claim 21, further comprising a stop feature associated with the shaft.

23. The cortical cap removal device according to claim 21, wherein the center point feature extends distally beyond the cutting element.

24. The cortical cap removal device according to claim 21, wherein the center point feature includes a surface treatment and/or surface feature that enhances engagement with a cortical cap.

25. The cortical cap removal device according to claim 21, wherein the cutting element defines a frustoconical geometry.

26. The cortical cap removal device according to claim 21, wherein the cutting element defines a compass single point or compass double point geometry.

27. The cortical cap removal device according to claim 21, wherein the cutting element defines an angled carver device.

28. The cortical cap removal device according to claim 27, wherein the angled carver device includes an angled carving blade.

29. The cortical cap removal device according to claim 21, further comprising cannulation.

30. The cortical cap removal device according to claim 29, further comprising an anchor spike or anchoring pin, and wherein the anchor spike or anchoring pin is configured and dimensioned to slide through the cannulation.

31. The cortical cap removal device according to claim 21, further comprising a ring blade.
